SEQUENCE LISTING (6 sequences)

SEQ ID NO: 1 - DNA, 6450 bp, Homo sapiens
  CDS (361)..(2148)
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 2 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 3 - DNA, 2011 bp, Homo sapiens
  CDS (419)..(2011)
tttcagtttctccagctgctggctttttggacacccactcccccgccaggaggcagttgcaagcgcggaggctgcgagaaataactgcctcttgaaacttgcagggcgaagagcaggcggcgagcgctgggccggggagggaccacccgagctgcgacgggctctggggctgcggggcagggctggcgcccggagcctgagctgcaggaggtgcgctcgctttcctcaacaggtggcggcggggcgcgcgccgggagaccccccctaatgcgggaaaagcacgtgtccgcattttagagaaggcaaggccggtgtgtttatctgcaagccattatacttgcccacgaatctttgagaacattataatgacctttgtgcctcttcttgcaaggtgttttctcagctgttatctcaagacatggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtga

SEQ ID NO: 4 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 5 - DNA, 537 bp, Homo sapiens
  CDS (361)..(537)
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcc

SEQ ID NO: 6 - protein, 59 aa, Homo sapiens
MTMTLHTKASGMALLHQIQGNELEPLNRPQLKIPLERPLGEVYLDSSKPAVYNYPEGAA

(19) United States
(12) Patent Application Publication
(10) Pub. No.: US 2003/0199472 A1
(43) Pub. Date: Oct. 23, 2003

(54) Adenovirus-mediated therapy for uterine fibroids

(75) Inventors: Ayman Al-Hendy, Houston, TX (US); Eun Jig Lee, Northbrook, IL (US); J. Larry Jameson, Winnetka, IL (US)

Correspondence Address: FULBRIGHT & JAWORSKI L.L.P., 600 CONGRESS AVE., SUITE 2400, AUSTIN, TX, 78701, US

(73) Assignees: Board of Regents, The University of Texas System; Northwestern University

(21) Appl. No.: 10/392,274

(22) Filed: Mar. 19, 2003

Related U.S. Application Data
(60) Provisional application No. 60/365,760, filed on Mar. 19, 2002.

(51) Int. Cl. C12Q 1/68; A61K 48/00

(52) U.S. Cl. 514/44; 435/6

(57) ABSTRACT

The present invention provides methods of treating and preventing uterine fibroids that is non-surgical, using a modified estrogen receptor gene delivered via an adenoviral vector. The modified estrogen receptor induced apoptosis in vitro and decreased tumor growth in vivo. The invention provides a major improvement above that of current available procedures for women having uterine fibroids, or in preventing fibroids in women at risk of having fibroids. The present invention further provides a safe means of treating fibroids and preserving fertility in young women, or maintaining pregnancy in pregnant women having fibroids.

The present invention claims priority to U.S. Provisional Patent Application Serial No. 60/365,760 filed on Mar. 19, 2002. The entire text of the above-referenced disclosure is specifically incorporated herein by reference. without disclaimer.

### BACKGROUND OF THE INVENTION

1. Field of the Invention

The present invention relates generally to the fields of molecular biology, gene therapy and gynecology. More particularly, it concerns down-regulation of estrogen responsive transcription in estrogen-responsive cells using modified estrogen receptors, an it further concerns adenoviral gene therapy for the treatment and prevention of uterine fibroids.

2. Description of Related Art

Clinical Importance of Uterine Fibroids

Uterine fibroids are the most common pelvic tumors in the United States, occurring in up to 77% of all women (Buttram and Reiter, 1981; Vollenhoven et al., 1990). They can cause some severe symptoms such as heavy, irregular, and prolonged menstrual bleeding and anemia. They also may cause pelvic discomfort, and bowel and bladder dysfunction from pressure. Fibroids have also been associated with infertility and recurrent abortion. These tumors tend to grow rapidly during pregnancy due to the influence of abundant estrogen available in the circulation, and can cause obstructed labor necessitating cesarean section, fetal malpresentation, and fetal anomalies, as well as postpartum hemorrhage secondary to uterine atony. Uterine fibroids account for 35% of all hysterectomies done in the United States with a huge economic impact on healthcare delivery system (Calson et al., 1993). Histologically, fibroids arise from a single uterine muscle cell, and they grow under the influence of local growth factors and sex hormones including estrogen and progesterone (Rein et al., 1995). Fibroids appear after menarche, proliferate and grow during the reproductive years, and stabilize or regress after menopause. They may regrow after hormone replacement therapy (Sener et al., 1996). The diagnosis of fibroids is based on patient signs and symptoms, followed by pelvic examination, demonstrating a pelvic mass, and confirmation by transabdominal or transvaginal ultrasonic measurements. The etiology is not clearly understood.

Few treatment options are currently available to women with symptomatic fibroids (Vilos, 2000) with the mainstay of treatment being surgery. Gonadotropin-releasing hormone agonists (GnRH-a) inhibit steroidogenesis and induce menopause and hence can reduce fibroid volume by 50% in 3 to 6 months. However, because of severe menopausal symptoms and irreversible bone loss (osteoporosis), these drugs cannot be used for prolonged periods of time. Fibroids tend to regrow after cessation of GnRH-a therapy and hence these agonists are not recognized as an effective treatment of fibroids. The two classical surgeries for treatment of uterine fibroids are myomectomy and hysterectomy (Vilos, 2000).

Myomectomy done either through a laparotomy or laparoscopy aims to remove the fibroid and conserve the uterus. This is usually attempted in young women desiring future fertility. Unfortunately, 95% of any type of myomectomy is followed by extensive pelvic adhesions that themselves can preclude future fertility. Additionally, if the fibroid penetrates the uterine cavity, any future pregnancy after myomectomy carries an increased risk of uterine rupture and delivery has to be accomplished by cesarean section.

Hysterectomy has been the mainstay for the treatment of fibroids. Between 1965 and 1987, more than 14 million hysterectomies were performed in the United States (Heelsom et al., 1993). Uterine fibroids account for approximately 67% of all hysterectomies performed among middle-aged women (Chryssikopoulos and Loghis, 1986). This surgical approach is extremely costly especially considering the long postoperative time away from work. Total, subtotal, vaginal, or laparoscopic hysterectomy can be done taking into account the patient's wishes and preferences. Although hysterectomy is a common and safe procedure, it carries a risk of major complications in 15% to 38% of cases (VeKaut, 1993). Such complications include postoperative hemorrhage, fever, or injury to adjacent organs. The risk of death is 0.5 per 1000 cases.

Two recent modalities have been developed for treatment of uterine fibroids: myolysis and uterine artery embolization. Myolysis refers to the technique where an attempt is made to disrupt or abolish the blood supply to the fibroid causing shrinkage using bipolar or monopolar electrosurgery (Vilos, 1997). It is only applicable if there are less than three fibroids present and/or the largest one measures less than 10 cm in diameter. The procedure is also not recommended for women who wish to get pregnant, since the risk of uterine rupture is very high (Vilos et al., 1998). Uterine artery embolization is a procedure done by radiologists and attempts to cut the blood supply to the fibroid (Ravina et al., 1995; Goodwin and Walker, 1998; Bradley et al., 1998; Hutchins and Berkowitz, 1999). The procedure is usually followed by severe pain requiring hospitalization for analgesia. No long-term follow up is available. Some concern about future fertility has been raised as well as the possibility of missing other fibroids or uterine malignancy. Close to 1% of women undergoing uterine artery embolism develop subsequent amenorrhea and menopause due to inadvertent impairment of ovarian function.

In summary, for a young woman with symptomatic fibroids who wants to preserve her fertility, there is currently no conservative and safe method of treatment that will manage her fibroids without compromising her subsequent chances of achieving a healthy and safe pregnancy.

### SUMMARY OF THE INVENTION

The present invention is based on the observation that modified estrogen receptors can be delivered and expressed in estrogen-responsive cells of the genitourinary tract to effect cell killing and to reduce tumor growth. Accordingly, the present invention concerns therapeutic and preventative methods and compositions for genitourinary conditions involving estrogen-responsive or estrogen-dependent cells.

Methods of the present invention include methods for treating an estrogen-dependent genitourinary condition in a patient and methods for inhibiting a leiomyoma cell. In some embodiments of the invention, methods include administering to the patient or to the cell an effective amount of an expression construct comprising a nucleic acid comprising a sequence encoding a modified estrogen receptor, wherein the sequence is under the control of a promoter. The term “estrogen-dependent” in the context of a disease or condition refers to a disease or condition that requires estrogen or the estrogen signal transduction pathway for its initiation, maintenance, and/or progression. Estrogen-dependent genitourinary conditions or diseases include those affecting the uterus, ovaries, cervix, and any other organ in the genitorurinary system. “Estrogen-dependent” in the context of a cell indicates the cell requires estrogen for it to continue living in its current state. The term “estrogen-responsive” indicates a cell or condition is affected by estrogen or other components of the estrogen signal transduction pathway. It may be understood that estrogen-dependent and estrogen-responsive cells express estrogen receptors when estrogen is present and/or transcribe genes whose promoters contain an estrogen-responsive element (ERE) when estrogen is present.

Estrogen-dependent uterine conditions or diseases include, but are not limited to, leiomyoma, adenomyosis, endometriosis, endometrial hyperplasia, leiomyosarcoma, tumor (benign or malignant), and dysfunctional uterine bleeding. In specific embodiments of the invention, the condition is leiomyoma or afflicts leiomyoma cells, or other slow growing gynecological conditions such as those of uterine origin. In still further embodiments, the leiomyoma is a submucous, intramural, or subserous fibroid.

In some methods of the invention, a patient in need of treatment or in need of a preventative regimen is first identified. A patient in need of treatment may be identified by a diagnosis (preliminary or confirmed) of the estrogen-dependent genitourinary condition. Accordingly, in some embodiments of the invention, a patient is identified in need of treatment by detecting a leiomyoma in the patient. Alternatively, a patient may be identified in need of compositions and methods of the invention by identifying the patient as one at risk for an estrogen-dependent genitourinary condition. In some embodiments, the patient has already been treated for a genitourinary condition, such as surgical elimination of a fibroid, and treatment is implemented to prevent additional fibroid growth or treat any fibroids not eliminated by surgery. Treatment (preventative or therapeutic) may be conducted in vivo or ex vivo.

The present invention involves compositions comprising modified estrogen receptors. A “modified estrogen receptor” refers to an estrogen receptor polypeptide that 1) has an amino acid sequence that differs from a wild-type estrogen receptor by at least one amino acid residue; 2) possesses at least one activity of a wild-type estrogen receptor; and 3) has at least one activity of the wild-type estrogen receptor that is reduced, eliminated, attenuated, weakened, compromised, or absent. Activities include: specific binding to a wild-type estrogen receptor (ability to homodimerize); specific binding to an ERE; specific binding to estrogen or estrogen derivatives or analogs; specific binding to growth factors, localizing in cytoplasm and nucleus; contributing to transcription of ERE-containing genes; specific binding to AP-1 complex or any components of AP-1 (for example, c-jun, Jun B, c-fos); or any other activity of the estrogen receptor, such that the effect of any estrogen receptor-specific activating substance, such as estrogen, is reduced by or by at least about 10, 20, 30, 40, 50, 60, 70, 80, 90, or 100%. The activity of a modified estrogen receptor is reduced if that activity is detectably less than the activity of the wild-type estrogen receptor (for example, less than 90%, 80%, 70%, 60, 50% or less percent than wild type). In some embodiments of the invention the modified estrogen receptor comprises, is at least, or is at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700 or more contiguous amino acids of a wild-type estrogen receptor. In still further embodiments, the wild-type receptor is SEQ ID NO:2 or SEQ ID NO:4.

Modified estrogen receptors may be created from estrogen receptor α or estrogen receptor β. The nucleic acid encoding an estrogen receptor may be from humans or any other mammal, and as identified by GenBank Accession NO. NM000125 (SEQ ID NO:1) and NM001437 (SEQ ID NO:3). of estrogen receptor α and estrogen receptor β respectively. Modified estrogen receptors may be created by randomly or specifically mutating a wild-type estrogen receptor encoding sequence or by identifying such a naturally occurring mutation. In some embodiments, the mutation affects DNA binding activity, dimerization, or transcriptional activation activity. Mutations may be insertions, deletions, or substitution. The mutations may introduce a frameshift and/or introduce a premature stop codon. In some embodiments, the modified estrogen receptor is produced as a result of a frameshift introduced at codon 554 (S554fs). In still further embodiments, the mutation is a point mutation. Alternatively, a mutation may involve more than one nucleotide. It may involve 1, 2, 3, 4, 5, 6, 7, 8, 9, 10, 15, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000 or more nucleotides, or at least or at most that many nucleotides. In some embodiments, the point mutation involves a substitution of leucine with a glutamine at codon 540 (L540Q).

In some embodiments of the invention, the mutation is a substitution in which a nonhomologous change is made, for example, when a charged residue is substituted for an uncharged residue, or vice versa. In other embodiments, the mutation is a deletion that results in a truncated receptor. In specific examples, the truncated receptor is ER1-536, which contains amino acids 1 through 536 and lacks the remaining 59 amino acids of the estrogen receptor. In other examples the truncated receptor is ER1-530, which is missing the last 65 amino acids of the wild-type protein. In some embodiments of the invention, a modified estrogen receptor has a mutation in a transactivation domain, in a DNA binding domain, or in a region mediating protein-protein interaction. A transactivation domain is the region of the polypeptide that is involved in transactivation of a gene through the ERE. A DNA binding domain refers to the region of the polypeptide that is involved in specific recognition and binding of DNA, including an ERE. A region mediating protein-protein interactions refers to the region of the polypeptide that is involved in specific binding of the estrogen receptor with polypeptides, such as another estrogen receptor molecule or AP-1, or components of AP-1.

In further methods of the invention, a modified estrogen receptor is provided to a cell by providing a viral vector as the expression construct comprising a nucleic acid encoding a modified estrogen receptor. In some embodiments of the invention, the viral vector includes, but is not limited to, an adenovirus vector, an adeno-associated virus vector, a herpesvirus vector, a lentivirus vector, a retrovirus vector, or a vaccinia virus vector. In specific embodiments, the viral vector is an adenovirus vector. In still further embodiments, the adenovirus vector encoding a modified estrogen receptor is Ad-ER1-536, which is also known as ER alpha 1-536. In still further embodiments, expression constructs of the invention include a promoter operatively linked to a nucleic acid encoding a modified estrogen receptor. In some embodiments, the promoter is a CMV IE promoter, modified CALC-I promoter (TCP), SV40 promoter, MMLV promoter, metallothenein II (MT II) promoter, or any estrogen-responsive promoter or promoter containing an ERE. In specific embodiments, the promoter is a CMV IE promoter, or a variation thereof, including basepairs 1-589 of that promoter.

In some methods of the invention, a patient is administered about 10³ to about 10¹⁵ viral particles, though the patient may be administered about or at least about 10³, 10⁴, 10⁵, 10⁶, 10⁷, 10⁸, 10⁹, 10¹⁰, 10¹¹, 10¹¹, 10¹², 10¹³, 10¹⁴, or 10¹⁵ viral particles. There may be multiple administrations, such as 2, 3, 4, 5, 6, 7, 8, 9, 10 or more administrations, which may be given hourly, daily, weekly, biweekly, monthly, bimonthly or annually. Furthermore, more than one different modified estrogen receptor may be administered to a patient or to a cell. Different modified estrogen receptors include those that have different mutations. In addition, a modified estrogen receptor a may be provided with a modified estrogen receptor β. The different modified estrogen receptors may be administered separately or they may be administered at the same time, on separate expression constructs or encoded by the same expression construct.

Compositions may be administered to the patient intrauterinely, intravaginally, intravenously, directly to the affected area, peritoneally, or regionally. In some embodiments, compositions are administered intrauterinely or intravaginally, which may involve using a catheter. In some embodiments, compositions are administered to the patient directly to the affected area. This may be accomplished by direct injection of the affected area. The term “effective amount” refers to the amount that is needed to achieve a particular goal, such as a treatment of a condition. In some embodiments, an effective amount refers to the amount needed to achieve a therapeutic benefit. In the context of the present invention, a “therapeutic benefit” refers to anything that promotes or enhances the well-being of the subject with respect to the medical treatment of her condition, which includes treatment of genitourinary diseases or conditions. A list of nonexhaustive examples of this includes extension of the subject's life by any period of time, decrease or delay in the development of the disease or condition, decrease in growth or size of a fibroid, decrease in number of fibroids, decrease of symptoms of condition or disease, increased fertility, reduction in fibroid growth, delay of recurrence, reduction in cell proliferation rate, and a decrease in pain to the subject that can be attributed to the subject's condition. In some embodiments, a leiomyoma cell is inhibited. In still further embodiments, a leiomyoma cell is inhibited, meaning the cell either undergoes apoptosis or has a reduced growth rate.

In some embodiments of the invention, methods further comprise removing all or part of a leiomyoma. The removal may occur before, after, or at the same time compositions of the invention are administered.

Methods of the invention further include inhibiting a leiomyoma cell or inducing a leiomyoma cell to undergo apoptosis. Inhibiting a cancerous cell includes slowing or halting the growth of the cancer cell, as well as inducing cell death. In addition to a treatment method, the present invention provides a way to study leiomyoma cells in vitro. Because of a bystander effect, a cell that is induced to undergo apoptosis may or may not be the cell administered compositions of the invention. A leiomyoma cell may be proximate to a transfected cell and be induced to undergo apoptosis. In some embodiments, the cell is in a patient.

Other methods of the invention involve preventing pregnancy in a female subject by administering an effective amount of an expression construct comprising a nucleic acid, under the control of a promoter, encoding a modified estrogen receptor. “Pregnancy” is understood to refer to the implantation of a fertilized egg for more than seven days. Embodiments discussed above are contemplated for use in this method. Thus, it is specifically contemplated that the expression vector may be a viral vector, as is described above. Furthermore, the modified estrogen receptor may be a modified estrogen receptor α or β. In some cases, the modified estrogen receptor has a mutation that affects DNA binding activity, transcriptional activation activity, dimerization activity, ligand binding activity, or growth hormone binding activity, binding activity to AP-1 or to a component of AP-1. In still further embodiments, the modified estrogen receptor is a dominant-negative estrogen receptor, such as ER1-536. Constructs may be administered directly to the uterine cavity, such as by injection or by using a catheter. Alternatively, it may be in the form of a suppository, or it may be administered orally, intravenuously, topically, or it may be administered to the subject as a patch in which the receptor is absorbed by the subject. The expression construct may be administered once or mulitple times. It is contemplated that it may be administered before, about the same time, or after ovulation in the female subject during that month's cycle. Compositions may also be administered or taken at various times during the month's cycle. Furthermore, other agents may be administered in combination with the modified estrogen receptor to prevent pregnancy. Such agents may be oral contraceptive pills or patch, the “day-after” pill, or other agents that inhibit or prevent pregnancy.

In addition to the use of a nucleic acid encoding a modified estrogen receptor, it is contemplated that in any of the embodiments discussed above with respect to nucleic acids, a modified estrogen receptor polypeptide instead may be employed.

Embodiments discussed with respect to one embodiment or example of the invention may be employed or implemented with respect to any other embodiment of the invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Ad-DNER constructs.

FIG. 2. Effect of Ad-DNER on the Growth of LM-1 cells.

FIG. 3. Caspase-3 activity in Ad-DNER treated ELT3 cells.

FIG. 4. Ad-DNER inhibits tumor development in vivo.

FIG. 5. Effect of AdER-DN treatment on subcutaneous fibroid tumor progression in nude mice. Direct intratumor injection of different treatments was performed on day 16 post-cell-implantation. AdER-DN treatment caused immediate overall arrest of tumor growth. The difference among treatment and control groups was highly significant (P=0.007). Results are mean±standard error of the mean.

FIG. 6. Nude mice with subcutaneous leiomyomas. Tumors treated with AdER-DN demonstrated arrest of tumor progression.

FIG. 7. BrdU incorporation of fibroid tissue under different treatment conditions in nude mice. Shown are representative BrdU-labeled fibroid sections counterstained with hematoxylin and eosin (×200) for each treatment option. Ad-LacZ (negative control), medium alone (negative control), or AdER-DN. As seen in the bar graph, significantly less BrdU staining, and therefore proliferation, was detected in tumors treated with AdER-DN (P<0.0001). Results are mean±standard deviation of the three different experiments.

FIG. 8. TUNEL reaction on fibroid tumor specimens from different treatment groups. Tissue samples of fibroid tumors collected from mice treated with Ad-LacZ, medium, or AdER-DN were processed for TUNEL assays. Fluorescent staining indicates TUNEL labeling, which signifies DNA fragmentation and apoptosis. As seen in the bar graph, significantly more apoptotic nuclei were detected in tumors treated with AdER-DN (P<0.0001). Results are mean±standard deviation of the three different experiments.

### DESCRIPTION OF ILLUSTRATIVE EMBODIMENTS

Uterine fibroids are the most common tumor in women in the United States. The present invention provides in vivo studies on a method for treating genitourinary conditions such as uterine fibroids. The uterus, a major target tissue for ovarian hormones, is composed of heterogeneous cell types such as stroma, luminal and glandular epithelia, and smooth muscle that undergo continuous synchronized changes of proliferation and differentiation in response to changes in levels of circulating estrogen and progesterone. Estrogen, by regulating estrogen target genes in a cell-specific manner, has different effects on different types of cells in the uterus.

The only previous work on gene therapy for fibroid tumors utilized a nonviral vector delivering a “suicide gene” expressing thymidine kinase, which converts a co-administered nucleoside analogue, ganciclovir, to its cytotoxic, phosphorylated form within transfected cells (Niu et al., 1998). The treatment described by Niu et al. demonstrated significant cell death of the human leiomyoma cells upon their transfection in vitro. In this study, estrogen was actually co-administered short term to promote leiomyoma cell growth and lead to more tumor cells being sensitive to the toxic phosphorylated ganciclovir. However, neither this study nor any subsequent studies reported in vivo data on gene therapy of uterine fibroids.

The present invention is the first indication that adenovirus can successfully infect human and rat leiomyoma cells. Leiomyomas are slow growing tumors, unlike breast cancer and pituitary cancer cells, and therefore, their ability to be infected by viruses was questionable. The present invention further contemplates adenoviral modified estrogen receptor therapy for the treatment of genitourinary conditions, which, in addition to leiomyomas, includes adenomyosis, endometriosis, endometrial hyperplasia or cancer.

Compositions and methods of the present invention provide the following advantages: 1) modified estrogen receptor (ER) compositions utilize the hormonal-dependency of cells, such as uterine fibroids, on estrogen to achieve their effect; 2) modified ER compositions provide safety for other adjacent organs that lack ER expression and would otherwise be harmed by inadvertent expression of these compositions; 3) further these compositions directly target the tumor; 4) modified ER compositions overcome the side effects of long-term exposure with chemotherapeutic agents; 5) modified ER compositions overcome the prior art in that inhibition of DNA polymerization, which occurs with thymidine kinase/ganciclovir system (TK/GCV), would be less effective than ER, which induces apoptosis and allows the immune system to attack the apoptotic cells and clear them; 6) these ER compositions are effective in vivo for the treatment of uterine fibroids; 7) modified ER compositions offers a safe, more effective, and novel nonsurgical treatment for women wanting to preserve fertility, or who are not fit for surgery; and circumvents drastic procedures such as hysterectomies.

I. Estrogen Receptor and Modified Estrogen Receptors

It has been demonstrated that estrogen stimulates and antiestrogens inhibit leiomyoma cell growth both in vitro and in vivo (Howe et al., 1995; Palomba et al., 2001). This growth-promoting effect of estrogen has also been demonstrated in several clinical reports and is mediated via estrogen receptors (ER) which have increased expression in fibroid tissues (Ichimura et al., 1998; Englund et al., 1998). Estrogen receptors (ERα and ERβ), belong to the large family of nuclear receptors. ERα and ERβ receptors both bind estrogen as well as other agonists and antagonists, and have distinct structural differences.

Two of the most interesting sites on the ER molecule are its ligand binding domain (LBD), otherwise known as AF-2, and its growth factor binding domain, otherwise known as AF-1. In addition, the DNA-binding domain (DBD) is responsible for binding at estrogen response elements (ERE) on the chromosome. ERα and ERβ, when complexed with estrogen, were shown to signal in opposite ways from an AP-1 site, with estrogen activating transcription in the presence of ERα and inhibiting transcription in the presence of ERβ.

A definitive role for ERα in the uterotrophic effects of E₂ was confirmed in adult female ERα knockout mice, where there is loss of estrogen responsiveness (Lubahn et al., 1993), as well as in mice with disruption of the estrogen-responsive ring finger protein gene (Orimo et al., 1999). ERβ is present in both endometrium and myometrium in several animal species (Matsuzaki et al., 1999; Wu et al., 2000; Sauders et al., 1997; Pelletier et al., 1999; Fujimoto et al., 1999; Wang et al., 1999), but its function in the uterus remains to be elucidated. ERβ levels in the uterus change during the menstrual cycle with the highest levels present during the proliferation phase (Matsuzaki et al., 1999) when estrogen and ERα levels are also at their peaks. ERβ has been found to act as a modulator of ERα-mediated gene transcription in the uterus.

The present invention therefore contemplates the use of a modified form of estrogen receptor as a therapeutic gene. Dominant negative forms of the ER have been suggested as a method to inactivate the ER. Several dominant negative ER mutants have been generated (Ince et al., 1993; Ince et al., 1995; Chien et al., 1999) which include: truncated receptors (ER1-530 and ER1-536, missing the last 65 and 59 amino acid residues, respectively), a point mutant (L540Q), and a frameshift mutant (S554fs). “Dominant negative mutants” refer to mutants that can act to override or inhibit the activity and/or expression of the wild-type molecules. Adenovirus-directed expression of the frame-shifted ER (S554fs) was shown to suppress the proliferation of ER-positive breast cancer cells (Lazennec et al., 1999). Adenovirus-mediated expression of a truncated receptor (ER1-536) has also been demonstrated to induce apoptosis in rat pituitary prolactinoma cells and inhibited tumor growth in nude mice (Lee et al., 2001). However, it should be noted that the breast cancer cells and the pituitary prolactinoma cells as in the above studies are not slow growing as are uterine fibroid cells.

The ER1-536 mutant seems to exert its growth-inhibiting effect by making inactive heterodimers with wild-type ER. These heterodimers could be unable to bind to the estrogen-responsive elements (ERE) in different growth-related genes or unable to activate transcription when bound to ERE (Ince et al., 1993).

The present invention provides an adenoviral vector carrying nucleic acid sequences encoding modified estrogen receptors to inhibit the growth of human and rat leiomyoma cells both in vitro and in vivo. The present invention further provides a novel nonsurgical approach to gene therapy of uterine fibroids. Since this treatment is localized to the tumor area itself and replication incompetent adenovirus is used, this therapy overcomes the prior art in the advantage of not interfering with ovulation or conception, or disturbing the progress of an ongoing pregnancy.

II. Gene Therapy

The present invention therefore provides in vitro, ex vivo, and in vivo gene therapy methods as an alternative and conservative treatment of uterine fibroids. Uterine fibroids are an attractive target for gene therapy because of several inherent biologic features. The disease is localized and well-circumscribed in the uterus, which simplifies targeting the treatment to the tumor by direct intratumor injection either under ultrasound guidance or using existing endoscopic procedures like laparoscopy or hystroscopy. Another favorable feature is that uterine fibroids are slow-growing tumors and marked clinical improvement of fibroid-related symptoms (e.g., irregular vaginal bleeding, pelvic pain, and infertility) does not necessitate complete resolution of the fibroid but rather a modest decrease in their size (Vilos, 1997). Unlike cancer gene therapy, achieving gene delivery into every single leiomyoma cell is not necessary to attain clinical improvement. This is a great advantage since with the current gene therapy vectors, it is extremely difficult to achieve 100% gene transfer in vivo (Niu et al, 1998).

III. Nucleic Acids of the Estrogen Receptor

The present invention concerns polynucleotides that are free from total genomic DNA and that are capable of expressing all or part of a protein or polypeptide. The polynucleotide may encode a peptide or polypeptide having all or part of the amino acid sequence of a wild-type or modified estrogen receptor. One embodiment of the present invention is to transfer the nucleic acids encoding the modified or truncated form of human estrogen receptor to induce apoptosis or inhibit growth of uterine fibroids.

Thus, in some embodiments of the present invention, the treatment of genitourinary conditions involves the administration of a therapeutic nucleic acid expression construct encoding a modified or truncated form of estrogen receptor to hyperproliferative cells. It is contemplated that the hyperproliferative cells take up the construct and express the therapeutic polypeptide encoded by nucleic acid, thereby inhibiting proliferation, restoring growth control to, or abrogating the hyperproliferative cells. Furthermore, it is contemplated that a soluble estrogen receptor released from transfected or transduced cells will be available locally and provide a bystander effect on neighboring tumor cells. Thus, it is contemplated further that the therapeutic estrogen receptor expression construct may be delivered to normal cells and the released bystander effects would further generate anti-tumor effects, particularly with respect to hyperproliferative cells that are estrogen dependent an/or express the estrogen receptor.

As used herein, the term “DNA segment” refers to a DNA molecule that has been isolated free of total genomic DNA of a particular species. Therefore, a DNA segment encoding a polypeptide refers to a DNA segment that contains wild-type, truncated, or modified polypeptide-coding sequences yet is isolated away from, or purified free from, total mammalian or human genomic DNA. Included within the term “DNA segment” are oligonucleotides and recombinant vectors, including, for example, plasmids, cosmids, phage, viruses, and the like.

As used in this application, the term “estrogen receptor polynucleotide” refers to a estrogen receptor-encoding nucleic acid molecule that has been isolated free of total genomic nucleic acid. Therefore, a “polynucleotide encoding estrogen receptor” refers to a DNA segment that contains wild-type (SEQ ID NO:1), mutant or truncated (SEQ ID NO:3), or polymorphic estrogen receptor polypeptide-coding sequences isolated away from, or purified free from, total mammalian or human genomic DNA. Therefore, for example, when the present application refers to the function or activity of a modified estrogen receptor or a “modified estrogen receptor polypeptide,” it is meant that the polynucleotide encodes a molecule whose amino acid sequence differs from wild-type and that it directly or indirectly inhibits, impedes, reduces, suppresses or abrogates transcriptional activity of the estrogen receptor.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e., two or more alleles exist) at a genetic locus in the individuals of a population. As used herein, “mutant” refers to a change in the sequence of a nucleic acid or its encoded protein, polypeptide, or peptide that is the result of recombinant DNA technology or the result of a mutation generated inside a cell that alters the physiology of that cell. “Mutant” includes “modified” sequences.

It also is contemplated that a particular polypeptide from a given species may be represented by natural variants that have slightly different nucleic acid sequences but, nonetheless, encode the same protein.

Similarly, a polynucleotide comprising an isolated or purified wild-type, polymorphic, or mutant polypeptide gene refers to a DNA segment including wild-type, polymorphic, or mutant polypeptide coding sequences and, in certain aspects, regulatory sequences, isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a wild-type or modified polypeptide may contain a contiguous nucleic acid sequence as set forth in SEQ. ID NO:1, SEQ. ID NO:3, or SEQ ID NO:5 encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

In particular embodiments, the invention concerns isolated DNA segments and recombinant vectors incorporating DNA sequences that encode a wild-type, truncated, or mutant estrogen receptor or estrogen receptor polypeptide or peptide that includes within its amino acid sequence a contiguous amino acid sequence in accordance with, or essentially corresponding to a native polypeptide. Thus, an isolated DNA segment or vector containing a DNA segment may encode, for example, a dominant negative estrogen receptor that can inhibit tumor growth and induce apoptosis. The term “recombinant” may be used in conjunction with a polypeptide or the name of a specific polypeptide, and this generally refers to a polypeptide produced from a nucleic acid molecule that has been manipulated in vitro or that is the replicated product of such a molecule.

In other embodiments, the invention concerns isolated DNA segments and recombinant vectors incorporating DNA sequences that encode a polypeptide or peptide that includes within its amino acid sequence a contiguous amino acid sequence in accordance with, or essentially corresponding to the polypeptide.

The nucleic acid segments used in the present invention, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, for example a mutated or truncated estrogen receptor polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides identical to or complementary to a particular gene, such as the wildtype estrogen receptor α (SEQ ID NO: 1) or wildtype estrogen receptor β (SEQ ID NO:3) or a modified estrogen receptor (SEQ ID NO:5) encoding nucleic acids. A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

The DNA segments used in the present invention encompass biologically functional equivalent modified polypeptides and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by a human may be introduced through the application of site-directed mutagenesis techniques, e.g., to introduce improvements to the antigenicity of the protein, to reduce toxicity effects of the protein in vivo to a subject given the protein, or to increase the efficacy of any treatment involving the protein.

Site-specific mutagenesis is a technique useful in the preparation of individual peptides, biologically functional equivalent or modified proteins, polypeptides or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired proteinaceous molecule. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

In certain other embodiments, the invention concerns isolated DNA segments and recombinant vectors that include within their sequence a contiguous nucleic acid sequence from that shown in SEQ ID NO:1, SEQ ID NO:3, or SEQ ID NO:5. This definition is used in the same sense as described above and means that the nucleic acid sequence substantially corresponds to a contiguous portion of that shown in SEQ ID NO:1, SEQ ID NO:3, or SEQ ID NO:5 and has relatively few codons that are not identical, or functionally equivalent, to the codons of SEQ ID NO:1, SEQ ID NO:3, or SEQ ID NO:5. The term “functionally equivalent codon” is used herein to refer to codons that encode the same amino acid, such as the six codons for arginine or serine, and also refers to codons that encode biologically equivalent amino acids. Codons preferred for use in humans, are well known to those of skill in the art (Wada et. al., 1990). Codon preferences for other organisms also are well known to those of skill in the art (Wada et al., 1990, included herein in its entirety by reference).

The various probes and primers designed around the nucleotide sequences of the present invention may be of any length. By assigning numeric values to a sequence, for example, the first residue is 1, the second residue is 2, etc., an algorithm defining all primers can be proposed:

n to n+y

where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

It also will be understood that this invention is not limited to the particular nucleic acid and amino acid sequences of SEQ ID NO:1, SEQ ID NO:3, or SEQ ID NO:5, as well as SEQ ID NO:2, SEQ ID NO: 4, or SEQ ID NO:6. Recombinant vectors and isolated DNA segments may therefore variously include the estrogen receptor coding regions themselves, coding regions bearing selected alterations or modifications in the basic coding region, or they may encode larger polypeptides that nevertheless include estrogen receptor-coding regions or may encode biologically functional equivalent proteins or peptides that have variant amino acids sequences.

If desired, one also may prepare fusion proteins and peptides, e.g., where the estrogen receptor—or its-coding regions are aligned within the same expression unit with other proteins or peptides having desired functions, such as for purification or immunodetection purposes (e.g., proteins that may be purified by affinity chromatography and enzyme label coding regions, respectively).

Encompassed by certain embodiments of the present invention are DNA segments encoding relatively small peptides, such as, for example, peptides of from about 15 to about 50 amino acids in length, and more preferably, of from about 15 to about 30 amino acids in length; and also larger polypeptides up to and including proteins corresponding to the full-length sequences set forth in SEQ ID NO:2. SEQ ID NO:4, or SEQ ID NO:6 or to specific fragments of SEQ ID NO:1, SEQ ID NO:3, SEQ ID NO:5 that correspond to differences as compared to the published sequence for estrogen receptor.

The term “a sequence essentially as set forth in SEQ. ID. NO:1” or “a sequence essentially as set forth in SEQ. ID. NO:1” means that the sequence substantially corresponds to a portion of SEQ. ID. NO:1 and has relatively few amino acids that are not identical to, or biologically functionally equivalent to, the amino acids of SEQ. ID. NO:2. It is contemplated that embodiments discussed with respect to a SEQ ID NO may be applied or implemented with respect to any other SEQ ID NO described herein.

IV. Proteinaceous Compositions

In certain embodiments, the present invention concerns novel compositions comprising at least one proteinaceous molecule, such as a modified estrogen receptor. As used herein, a “proteinaccous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater continuous amino molecule residues, and any range derivable therein of SEQ. ID. NO: 2, SEQ. ID. NO: 4, or SEQ ID NO:6.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be known to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

1. Functional Aspects of Dominant Negative Estrogen Receptor

The present invention concerns estrogen receptor, particularly a modified estrogen receptor (ER). The modified ER of the present invention has an anti-proliferative effect on a cell expressing estrogen receptors and/or is dependent on estrogen for growth. This modified ER promotes apoptosis in cells, or effects a reduction of tumors that are estrogen dependent, such as uterine fibroids. Thus, when the present application refers to the function or activity of a modified ER gene, it is meant that the molecule in question is unable to support estrogen-responsive gene transcription, which may include the ability to inactivate any wild-type estrogen receptor. Furthermore, the function or activity of a modified ER refers to its ability to affect DNA binding activity, transcriptional activation activity, dimerization activity, ligand binding activity, or its ability to bind AP-1 or its component(s).

When the present application refers to the function or activity of a modified estrogen receptor (ER) or a “modified ER polypeptide,” one of ordinary skill in the art would understand that this includes, for example, a mutant or truncated molecule with the ability to suppress, impede, inhibit, or reduce cell proliferation. On the other hand, when the present invention refers to the function or activity of a “modified ER gene,” one of ordinary skill in the art would further understand that this includes, for example, a molecule with the ability to induce apoptosis or an ability to promote cell death in a cell that expresses ER and/or is estrogen dependent.

Other phenotypes that may be considered to be associated with the expression of mutated, truncated or modified ER genes are the downregulation, inhibition, suppression or inactivation of transcription of ER associated genes. Additional phenotypes may include but are not limited to changes in angiogenesis, adhesion, migration, cell-cell signaling, cell growth, cell proliferation, density-dependent growth, anchorage-dependent growth or the inhibition, reduction, suppression of hyperproliferative diseases or genitourinary conditions such as uterine fibroids, endometriosis, adenomyosis, endometrial hyperplasia, or cancer.

Determination of the function or activity of a mutant, truncated or modified ER may be achieved using assays familiar to those of skill in the art. For example, the function of a modified gene can be identified by transferring the gene, or a variant thereof, into cells that are estrogen dependent and/or express estrogen receptors and assaying these cells for growth inhibition and/or apoptosis. Alternatively, transcriptional activity of the modified ER can be analyzed such as its ability to dimerize, to bind the estrogen response element (ERE), to activate transcription of the ERE containing promoters, to bind estrogen and/or bind AP-1 or its components (Jun, Fos).

2. Variants of Dominant Negative Estrogen Receptor

Amino acid sequence variants of the polypeptides of the present invention can be substitutional, insertional or deletion variants. Several ER mutants have been generated such as by truncation (ER1-530 and ER1-536, missing the last 65 or 59 amino acid residues), point mutation (L540Q) and frameshift mutations (S554fs). In particular embodiments, the present invention concerns a modified ER such as the ER1-536 mutant.

Deletion variants lack one or more residues of the native protein. Another common type of deletion variant is one which inactivates binding to the estrogen-responsive elements (ERE). Insertional mutants typically involve the addition of material at a non-terminal point in the polypeptide. This may include the insertion of an immunoreactive epitope or simply a single residue. Terminal additions, called fusion proteins, are discussed below.

Insertions and deletions involving one or two base pairs (or a number of base pairs that are not multiples of three) can have devastating consequences to the polypeptide encoded by the gene because translation of the gene is “frameshifted.” For example, by shifting the reading frame one nucleotide to the right, the same sequence of nucleotides encodes a different sequence of amino acids. The mRNA is translated in new groups of three nucleotides however, the protein specified by these new codons is unlikely to function properly. Frameshifts change multiple amino acids and often create new stop codons thereby generating nonsense mutations. With a nonsense mutation, the new nucleotide changes a codon that specified an amino acid to one of the stop codons (TAA, TAG, or TGA). Therefore translation, of the messenger RNA transcribed from this mutant gene prematurely stops. The earlier in the gene that a nonsense mutation occurs, the more truncated the protein product and the more likely that it will be unable to function.

Substitutions are changes to an existing amino acid. For example, an A-T may be replaced with G-C. When one or more amino acids are replaced by a new one, a point mutation occurs. Since the genetic code is read three steps at a time, point mutations can change at most one amino acid in a protein. Point mutations create genetic variation by creating new alleles. Natural selection operates on this variability by selecting the best alleles.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine or histidine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. On the other hand, the substitution of a T for a C at a specific nucleotide can converts for example, a glutamine codon (CAG) to a stop codon (TAG).

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

The following is a discussion based upon changing the amino acids of a protein, such as an estrogen receptor, to create a mutated, truncated, or modified protein. For example, as in the present invention, certain amino acids may be substituted for other amino acids in the estrogen receptor resulting in growth inhibition of estrogen dependent tumors, by affecting or down-modulating: dimerization of the ER; ligand binding activity; DNA binding; binding to the estrogen-responsive elements (ERE), or by activation of transcription from the ERE. Since it is the interactive capacity and nature of a protein that defines that protein's biological functional activity, certain amino acid substitutions can be made in a protein sequence, and in its underlying DNA coding sequence, thereby producing a mutated, truncated or modified protein. It is thus contemplated by the inventors that various changes may be made in the DNA sequences of genes which effectively alter their biological utility or activity, as is discussed below.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that for amino acids positioned in the homologous region of nucleotide and encodes for the protein or polypeptide, the substitution of pairs of homologous and non-homologous amino acids can be made effectively on the basis of polarity. Non-homologous amino acids may be conservatively substituted with a member of the same polarity group as defined below: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (See e.g., Johnson, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of the truncated or mutant estrogen receptor but with altered and even improved characteristics.

3. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

V. Methods of Gene Transfer

Native and modified polypeptides may be encoded by a nucleic acid molecule comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1996, both incorporated herein by reference. In addition to encoding a modified polypeptide such as modified gelonin, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targeting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Viral Vector-Mediated Transfer

The dominant negative estrogen receptor nucleic acids are incorporated into an adenoviral infectious particle to mediate gene transfer to a cell. Additional expression constructs encoding other therapeutic agents as described herein may also be transferred via viral transduction using infectious viral particles, for example, by transformation with an adenovirus vector of the present invention as described herein below. Alternatively, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Though adenovirus is exemplified, the present methods may be advantageously employed with other viral vectors, as discussed below.

The present invention provides a method for using adenoviral vectors to deliver the therapeutic gene instead of nonviral methods. Adenovirus has the advantage of being highly efficient in transfecting several cell types, as well as supporting high levels of gene targeting to the nucleus, resulting in significant gene expression (Hallenbeck and Stevenson, 2000). Also adenovirus stocks can be prepared to high concentrations, which will allow delivery of large amounts of viral particles in finite volumes (Kozarsky and Wilson, 1993).

i. Adenovirus

Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5 tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al, 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al, 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

ii. Retrovirus

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5 and 3 ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

iii. Adeno-Associated Viruses

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et al., 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1996; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1996; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

iv Other Viral Vectors

Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

2. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5 non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 1 lists several elements/promoters that may be employed, in the context of the present invention, to regulate the expression of a gene. This list is not intended to be exhaustive of all the possible elements involved in the promotion of expression but, merely, to be exemplary thereof. Table 2 provides examples of inducible elements, which are regions of a nucleic acid sequence that can be activated in response to a specific stimulus.

TABLE 1
Promoter and/or Enhancer

| Promoter/ | |
|---|---|
| Enhancer | References |
| Immuno- | Banerji et al., 1983; Gilles et al., 1983; Grosschedl et |
| globulin Heavy | al., 1985; Atchinson et al., 1986, 1987; Imler et al., |
| Chain | 1987; Weinberger et al., 1984; Kiledjian et al., 1988; |
| | Porton et al.; 1990 |
| Immuno- | Queen et al., 1983; Picard et al., 1984 |
| globulin Light | |
| Chain | |
| T-Cell | Luria et al., 1987; Winoto et al., 1989; Redondo et al.; |
| Receptor | 1990 |
| HLA DQ a | Sullivan et al., 1987 |
| and/or DQ | |
| -Interferon | Goodbourn et al., 1986; Fujita et al., 1987; Goodbourn |
| | et al., 1988 |
| Interleukin-2 | Greene et al., 1989 |
| Interleukin-2 | Greene et al., 1989; Lin et al., 1990 |
| Receptor | |
| MHC Class II | Koch et al., 1989 |
| 5 | |
| MHC Class II | Sherman et al., 1989 |
| HLA-DRa | |
| -Actin | Kawamoto et al., 1988; Ng et al.; 1989 |
| Muscle | Jaynes et al., 1988; Horlick et al., 1989; Johnson et al., |
| Creatine | 1989 |
| Kinase (MCK) | |
| Prealbumin | Costa et al., 1988 |
| (Transthyretin) | |
| Elastase I | Omitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| -Fetoprotein | Godbout et al., 1988; Campere et al., 1989 |
| t-Globin | Bodine et al., 1987; Perez-Stable et al., 1990 |
| -Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsh et al., 1990 |
| Adhesion | |
| Molecule | |
| (NCAM) | |
| ₁-Antitrypain | Latimer et al., 1990 |
| H2B (TH2B) | Hwang et al., 1990 |
| Histone | |
| Mouse and/or | Ripe et al., 1989 |
| Type I | |
| Collagen | |
| Glucose- | Chang et al., 1989 |
| Regulated | |
| Proteins | |
| (GRP94 and | |
| GRP78) | |
| Rat Growth | Larsen et al., 1986 |
| Hormone | |
| Human Serum | Edbrooke et al., 1989 |
| Amyloid A | |
| (SAA) | |
| Troponin I | Yutzey et al., 1989 |
| (TN I) | |
| Platelet- | Pech et al., 1989 |
| Derived | |
| Growth Factor | |
| (PDGF) | |
| Duchenne | Klamut et al., 1990 |
| Muscular | |
| Dystrophy | |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; |
| | Katinka et al., 1980, 1981; Tyndell et al., 1981; Dandolo |
| | et al., 1983; de Villiers et al., 1984; Hen et al., 1986; |
| | Satake et al., 1988; Campbell et al., 1988 |
| Retroviruses | Kriegler et al., 1982, 1983; Levinson et al., 1982; |
| | Kriegler et al., 1983, 1984a, b, 1988; Bosze et al., 1986; |
| | Miksicek et al., 1986; Celander et al., 1987; Thiesen |
| | et al., 1988; Celander et al., 1988; Chol et al., 1988; |
| | Reisman et al., 1989 |
| Papilloma | Campo et al., 1983; Lusky et al., 1983; Spandidos and |
| Virus | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B | Bulla et al., 1986; Jameel et al., 1986; Shaul et al., 1987; |
| Virus | Spandau et al., 1988; Vannice et al., 1988 |
| Human | Muesing et al., 1987; Hauber et al., 1988; Jakobovits |
| Immuno- | et al., 1988; Feng et al., 1988; Takebe et al., 1988; |
| deficiency | Rosen et al., 1988; Berkhout et al., 1989; Laspia et al., |
| Virus | 1989; Sharp et al., 1989; Braddock et al., 1989 |
| Cyto- | Weber et al., 1984; Boshart et al., 1985; Foecking et al., |
| megalovirus | 1986 |
| (CMV) | |
| Gibbon Ape | Holbrook et al., 1987; Quinn et al., 1989 |
| Leukemia | |
| Virus | |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; Searle |
| | | et al., 1985; Stuart et al., |
| | | 1985; Imagawa et al., 1987, |
| | | Karin et al., 1987; Angel |
| | | et al., 1987b; MeNeall et al., |
| | | 1989 |
| MMTV (mouse | Glucocorticoids | Huang et al., 1981; Lee |
| mammary tumor | | et al., 1981; Majors et al., |
| virus) | | 1983; Chandler et al., 1983; |
| | | Lee et al., 1984; Ponta et al., |
| | | 1985; Sakai et al., 1988 |
| -Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | Hug et al., 1988 |
| | Newcastle Disease | |
| | Virus | |
| GRP78 Gene | A23187 | Resendez et al., 1988 |
| -2-Macroglobulin | IL-6 | Kunz et al., 1989 |
| Vimentin | Serum | Rittling et al., 1989 |
| MHC Class I Gene | Interferon | Blanar et al., 1989 |
| H-2b | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996), and the SM22 promoter.

Also contemplated as useful in the present invention are the dectin-1 and dectin-2 promoters. Additional viral promoters, cellular promoters/enhancers and inducible promoters/enhancers that could be used in combination with the present invention are listed in Tables 1 and 2. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest. Alternatively, a tissue-specific promoter for cancer gene therapy (Table 3) or the targeting of tumors (Table 4) may be employed with the nucleic acid molecules of the present invention.

TABLE 3
Candidate Tissue-Specific Promoters for Cancer Gene Therapy

| | Cancers in which | Normal cells in which |
|---|---|---|
| Tissue-specific promoter | promoter is active | promoter is active |
| Carcinoembryonic antigen | Most colorectal | Colonic mucosa; gastric |
| (CEA)* | carcinomas; 50% of lung | mucosa; lung epithelia; eccrine |
| | carcinomas; 40-50% of | sweat glands; cells in testes |
| | gastric carcinomas; most | |
| | pancreatic carcinomas; | |
| | many breast carcinomas | |
| Prostate-specific antigen | Most prostate carcinomas | Prostate epithelium |
| (PSA) | | |
| Vasoactive intestinal peptide | Majority of non-small cell | Neurons; lymphocytes; mast |
| (VIP) | lung cancers | cells; eosinophils |
| Surfactant protein A (SP-A) | Many lung | Type II pneumocytes; Clara |
| | adenocarcinomas cells | |
| Human achaete-scute | Most small cell lung | Neuroendocrine cells in lung |
| homolog (hASH) | cancers | |
| Mucin-1 (MUC1)** | Most adenocarcinomas | Glandular epithelial cells in |
| | (originating from any | breast and in respiratory, |
| | issue) | gastrointestinal, and |
| | | genitourinary tracts |
| Alpha-fetoprotein | Most hepatocellular | Hepatocytes (under certain |
| | carcinomas; possibly many | conditions); testis |
| | testicular cancers | |
| Albumin | Most hepatocellular | Hepatocytes |
| | carcinomas | |
| Tyrosinase | Most melanomas | Melanocytes; astrocytes; |
| | | Schwann cells; some neurons |
| Tyrosine-binding protein | Most melanomas | Melanocytes; astrocytes, |
| (TRP) | | Schwann cells; some neurons |
| Keratin 14 | Presumably many | Keratinocytes |
| | squamous cell carcinomas | |
| | (e.g.: Head and neck | |
| | cancers) | |
| EBV LD-2 | Many squamous cell | Keratinocytes of upper digestive |
| | carcinomas of head and | Keratinocytes of upper digestive |
| | neck | tract |
| Glial fibrillary acidic protein | Many astrocytomas | Astrocytes |
| (GFAP) | | |
| Myelin basic protein (MBP) | Many gliomas | Oligodendrocytes |
| Testis-specific angiotensin- | Possibly many testicular | Spermatazoa |
| converting enzyme (Testis- | cancers | |
| specific ACE) | | |
| Osteocalcin | Possibly many | Osteoblasts |
| | osteosarcomas | |

TABLE 4
Candidate Promoters for Use with a Tissue-Specific Targeting
of Tumors

| | Cancers in which | Normal cells in which |
|---|---|---|
| Promoter | Promoter is active | Promoter is active |
| E2F-regulated | Almost all cancers | Proliferating cells |
| promoter | | |
| HLA-G | Many colorectal | Lymphocytes; monocytes; |
| | carcinomas; many | spermatocytes; trophoblast |
| | melanomas; possibly many | |
| | other cancers | |
| FasL | Most melanomas; many | Activated leukocytes: |
| | pancreatic carcinomas; | neurons; endothelial cells; |
| | most astrocytomas possibly | keratinocytes; cells in |
| | many other cancers | immunoprivileged tissues; |
| | | some cells in lungs, |
| | | ovaries, liver, and prostate |
| Myc-regulated | Most lung carcinomas | Proliferating cells (only |
| promoter | (both small cell and non- | some cell-types): mammary |
| | small cell); most colorectal | epithelial cells (including |
| | carcinomas | non-proliferating) |
| MAGE-1 | Many melanomas; some | Testis |
| | non-small cell lung | |
| | carcinomas; some breast | |
| | carcinomas | |
| VEGF | 70% of all cancers | Cells at sites of |
| | (constitutive | neovascularization (but |
| | overexpression in many | unlike in tumors, expres- |
| | cancers) | sion is transient, less |
| | | strong, and never |
| | | constitutive) |
| BFGF | Presumably many different | Cells at sites of ischemia |
| | cancers, since bFGF | (but unlike tumors, expres- |
| | expression is induced by | sion is transient, less |
| | ischemic conditions | strong, and never |
| | | constitutive) |
| COX-2 | Most colorectal | Cells at sites of |
| | carcinomas; many lung | inflammation |
| | carcinomas; possibly many | |
| | other cancers | |
| IL-10 | Most colorectal | Leukocytes |
| | carcinomas; many lung | |
| | carcinomas; many | |
| | squamous cell carcinomas | |
| | of head and neck; possibly | |
| | many other cancers | |
| GRP78/BiP | Presumably many different | Cells at sites of ishemia |
| | cancers, since GRP7S | |
| | expression is induced by | |
| | tumor-specific conditions | |
| CarG elements | Induced by ionization | Cells exposed to ionizing |
| from Egr-1 | radiation, so conceivably | radiation; leukocytes |
| | most tumors upon | |
| | irradiation | |

VI. Pharmaceutical Composition and Routes of Adminstration

In an embodiment of the present invention, a method of treatment for genitourinary conditions such as uterine fibroids, by the delivery of an adenoviral encoded modified estrogen receptor (or the modified estrogen receptor as a polypeptide) is contemplated. Other methods of the invention include the prevention of pregnancy. Uterine fibroids that are most likely to be treated in the present invention are those that are estrogen dependent or express the estrogen receptor. An increase in estrogen receptor expression or activity is considered to be related to the promotion or maintenance of unregulated growth control. Examples of genitourinary conditions contemplated for treatment include leiomyomas, adenomyosis, endometriosis, endometrila hyperplasia or cancer and any other hyperproliferative diseases that may be treated by altering the activity of estrogen receptor.

An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

Preferably, patients will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

1. Routes of Administration

To induce apoptosis, inhibit cell growth, inhibit metastasis, decrease tumor or tissue size and otherwise reverse or reduce the malignant phenotype of tumor cells, using the methods and compositions of the present invention, one would generally contact a hyperproliferative cell or tumor with the therapeutic compound such as a polypeptide or an expression construct encoding a polypeptide. To prevent pregnancy, a cell that is estrogen-responsive and involved in ovulation or implantation, is contacted with compositions of the invention. Uterine cells are specific targets in some embodiments of the invention.

The routes of administration will vary, naturally, with the location and nature of the target, and include, e.g., intrauterine, transdermal, parenteral, intravenous, intramuscular, subcutaneous, percutaneous, intratracheal, intraperitoneal, intratumoral, perfusion, lavage, and direct injection, or via catheter.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising an adenoviral modified estrogen receptor. The perfusion may be continued post-resection, for example, by leaving a catheter implanted at the site of the surgery. Periodic post-surgical treatment also is envisioned.

Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, disease progression, and health and age of the patient. Obviously, certain types of tumor will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles (vp) for a viral construct. Unit doses range from 10³, 10⁴, 10⁵, 10⁶, 10⁷, 10⁸, 10⁹, 10¹⁰, 10¹¹, 10¹², 10¹³ pfu and higher. Alternatively, depending on the kind of virus and the titer attainable, one will deliver 1 to 100, 10 to 50, 100-1000, or up to about 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ or higher infectious viral particles (vp) to the patient or to the patient's cells.

2. Injectable Compositions and Other Formulations

The preferred method for the delivery of an expression construct encoding all or part of a estrogen receptor or of the modified estrogen polypeptide to uterine fibroid tumors in the present invention is via intratumoral injection. However, the pharmaceutical compositions disclosed herein may alternatively be administered to uterine cells generally in the follow ways: directly, locally, topically, intrauterinely, intravenously, intravaginally, intraperitoneally, or intraregionally.

Injection of nucleic acid constructs may be delivered by syringe or any other method used for injection of a solution, as long as the expression construct can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vaccuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules, suppositories, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

VII. Combination Therapies with Modified Estrogen Receptors

In order to increase the effectiveness of a treatment with the compositions of the present invention, such as an adenoviral modified estrogen receptor, or expression construct coding therefor, it may be desirable to combine these compositions with other therapies effective in the treatment of uterine fibroids, such as anti-cancer agents, or surgery. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that ER therapy could be used similarly in conjunction with chemotherapeutic, radiotherapeutic, or other biological intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, where several days (2, 3, 4, 5, 6 or 7) to several weeks (1, 2, 3, 4, 5, 6, 7 or 8) lapse between the respective administrations.

Various combinations may be employed; a modified estrogen receptor is “A” and the secondary anti-cancer agent, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic expression constructs of the present invention to a patient will follow general protocols for the administration of the anti cancer therapy, taking into account the toxicity, if any, of the vector. It is expected that the treatment cycles would be repeated as necessary. It also is contemplated that various standard therapies, as well as surgical intervention, may be applied in combination with the described adenoviral therapy.

1. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery), laparascopic surgery and harmonic scalpel surgery. It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

2. Hormonal Therapy

Because fibroids grow in response to the female hormone estrogen, anti-estrogen hormones such as progesterone can shrink fibroids and may result in dramatic improvement in symptoms. Hormonal therapy is most useful in shrinking fibroids prior to surgery. The present invention therefore contemplates that hormonal therapy may be used with the present invention in treating and preventing uterine fibroids. Hormonal therapy may include a prescription for birth-control pills or other hormonal therapy, or the use of non-steroidal anti-inflammatory drugs (NSAIDs), such as ibuprofen or naproxen sodium. Aggressive hormonal therapy may employ Lupron. Lupron is a GNRH agonist that blocks ovarian estrogen production, is non-invasive, shrinks fibroids, and often improves symptoms. Other hormonal therapies contemplated with the present invention may include androgen, RU-486, and gestrinone. Additionally, a new drug, prifenidone which blocks a chemical that helps fibroids grow may also be employed with the present invention.

3. Other Gene Therapy

Other gene therapies may also be combined with the present invention. These include but are not limited to apoptosis promoting molecules such as the Bcl-2 family members that function to promote cell death such as Bax, Bak, Bik, Bim, Bid, Bad, Mtd, Bcl-XS and Harakiri.

The caspases such as caspase-3, caspase-7 and caspase-9 are known to play critical roles as executioners of apoptosis. Therefore, caspase gene therapy may also be used in combination with the present invention to further promote cell death or tumor reduction. It is further contemplated that agents such as the TNF family members which are well known in the art, may also be employed to further promote cell death of tumor cells with the present invention. Tumor necrosis factor-related apoptosis-inducing ligand (TRAIL/Apo2L) which activates apoptosis in numerous cancers without toxicity to normal cells, and Fas-ligand are two such TNF family members. Other gene therapies that may also be employed with the present invention include tumor suppressor genes such as E1A gene and p53 which can function by inducing apoptosis and inhibiting metastasis.

The methods by which to employ other gene therapy with that of the present invention are well known to those of skill in the art. All of the above methods may further employ adenoviruses in targeting pathways that are involved in mediating cell kill in tumor cells.

4. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

5. Radiotherapy

Other factors that cause DNA damage and have been used extensively in treating gynecological tumors is further contemplated for used in the present invention in treating uterine tumors. These include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Experimental Procedures

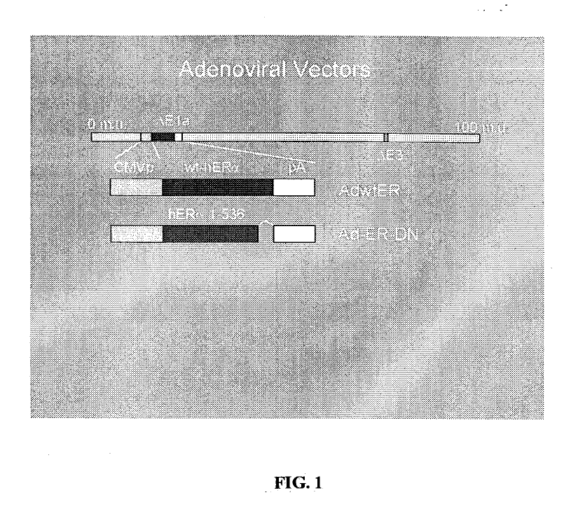

Preparation and amplification of recombinant adenovirus with dominant negative estrogen receptor: Adenoviral vectors carry the dominant negative ER mutant ER1-536 (Ad-ER also identified as Ad-DNER, Ad-ER-DN, AdER-DN, or Ad-ER1-536 or ER1-536; FIG. 1). The production of recombinant replication-deficient adenoviral vectors has been previously described (Chen et al., 1994; Bett et al., 1993) and is incorporated herein in its entirety. The 2.8-kbp Bgl II/Bam HI fragment containing the HSV-tk gene and poly(A) tail was inserted into the Bam HI site of the plasmid pADL.1/RSV, which were obtained by insertion of the Rous sarcoma virus long terminal repeat (RSV-LTR) promoter into the Xba I and Cla I sites of pXCJL. 1. In the resulting plasmid pADL.1/RSV-tk the HSV-tk gene is under the transcriptional control of RSV-LTR. To generate a recombinant adenovirus, pADL.1/RSV-tk and pJM17, a plasmid containing the complete adenovirus genome, were cotransfected into the 293 transformed human kidney cell line by calcium phosphate precipitation. Recombinant adenovirus was isolated from a single plaque, expanded in the 293 cell line, and purified by double cesium gradient ultracentrifugation as described in Graham et al., 1991.

The procedure as pertaining to the present invention is as follows:—Adenovirus carrying the dominant negative ER mutant ER1-536 (Ad-ER) was used to infect the adenovirus permissive human cell line 293. Forty 150-mm dishes of 293 cells were prepared and infected with Ad-ER at 1 to 10 PFU/cells. Cells were incubated at 37° C. and 5% CO₂ until signs of cytopathic effect were detected. When CPE is nearly complete (i.e., most of the cells are rounded but not yet detached), cells were harvested by scraping them off the dish. Cells were disrupted by three cycles of freezing (−70° C.) and thawing (37° C.), and the crude virus stock was titrated. After two cycles of cesium chloride ultracentrifugation, the purified virus was stored in the smallest possible volume aiming at stocks of about 10¹¹ PFU/mL.

Preparation of human leiomyoma cells: Samples of uterine leiomyomas were obtained from patients undergoing hysterectomy at the University of Texas Medical Branch. These samples were used in two different ways: first to obtain small fibroid tissue blocks 2 to 3 mm³ by cutting them under dissecting microscope. Second, human leiomyoma cells were prepared from these samples using the method published by Rauk and colleagues (Rauk et al., 1995).

This method is described as follows: Tissue samples were placed in modified Hanks' balanced salt solution (500 mL of calcium- and magnesium-free Hanks' balanced salt solution with 5 mL of heparin [1000 U/mL], 5 mL of gentamicin [50 mg/mL], and 5 mL of penicillin G-streptomycin [10,000 U/mL and 10,000 μg/mL]) and kept at 4° C. before processing. Using sterile technique, the tissue samples were minced, washed with Earle's balanced salt solution, and placed in a 15-mL conical tube and centrifuged at 500 g for 5 minutes. The pellets were resuspended in 5 mL of 0.125% trypsin solution and incubated at 37° C. for 15 minutes. Tissues were centrifuged at 500 g for 5 minutes and resuspended in 5 mL of collagenase type II solution (Worthington, Freehold, N.J.), 8 mg in 12.5 mL of Earle's balanced salt solution. The suspension was incubated at 37° C. for 2 to 3 hours with occasional pipetting. The suspension was passed through fine-mesh gauze, and individual cells were collected by centrifugation at 500 g for 5 minutes. The cells were washed twice with 10% minimal essential medium with nonessential amino acids, sodium bicarbonate (26 mmol/L), pyruvate (1 mmol/L), gentamicin (50 μg/mL), penicillin G (100 U/mL), streptomycin (100 μg/mL), L-glutamine (2 mmol/L), and charcoal-stripped fetal calf serum (10% [vol/vol]). The cells were then plated at 5×10⁵ cells in 25 cm² flasks and maintained at 37° C. in humidified 5% carbon dioxide. The medium was changed every 2 to 3 days, and the cells were subcultured at confluence after 5 to 7 days. The cells were identified at each passage as smooth muscle cells by means immunohistochemical staining with a monoclonal anti-alpha-smooth muscle actin antibody (Sigma, St. Louis, Mo.).

### Example 2

### Adenovirus Effectively Infects Human Leiomyoma Cells

Human leiomyoma cells were established as described in the methods section. Cells were infected with adenovirus carrying β-galactosidase gene. Successfully infected cells were blue after staining with X-gal stain.

### Example 3

### Adenovirus Infects Human Uterine Fibroid Explants

Samples of human fibroid tisues were collected at the time of hysterectomy and incubated with adenovirus carrying β-galactosidase gene as described in the methods section. The nucleus stained blue in smooth muscle cells of the fibroid growth. The tissues were counterstained with H&E (×400).

### Example 4

### Adenovirus with Dominant Negative Estrogen Receptor Induces Cell Death in Human Leiomyoma Cells

Human leiomyoma cells were infected with adenovirus carrying dominant negative estrogen receptor gene (Ad-ER) as described in the methods section. At MOI of 3.5 PFU/cell, all leiomyoma cells showed cell death 1 week after virus infection, measured by trypan blue exclusion test. The appearance of apoptosis vesicles in cells occured 5 days after treatment with Ad-ER with 40-50% of the nuclei showing positive TUNEL. Cells treated with adenovirus carrying the marker gene, β-galactosidase, appeared healthy.

### Example 5

### In Vitro Transfection of Human Leiomyoma Cells and Human Fibroid Tissue with Ad-ER Virus

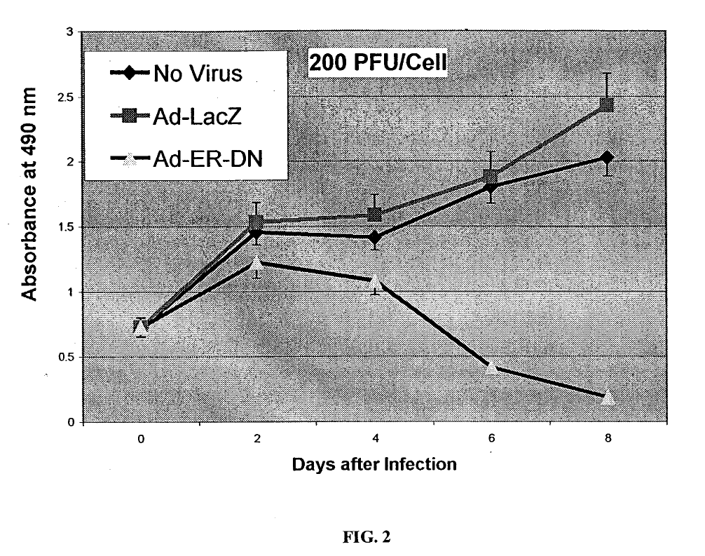

Since there were no published studies on the ability of adenoviral vector to infect human leiomyoma cells (HLC), an adenoviral vector expressing a marker gene was first tested. Ad-LacZ expresses β-galactosidase gene. Therefore, cells successfully transfected with this vector were identified by the presence of blue nuclei after reaction with a chromogenic substrate, 5-bromo-4-chloro-3-indolyl-β-D-galactoside. This vector was used as a reporter gene for monitoring transfection efficiency. The in vitro ability of Ad-LacZ to infect HLC as well as fresh human fibroid tissue cubes were assessed (FIG. 2). The cells were grown to 50% confluence in triplicates in 35-mm wells. The Ad-LacZ virus stock was diluted to 1, 10, 100, or 500 PFU/cell in culture medium and 1 mL of the virus suspension was placed in each well after removal of the medium. The virus was left in contact with the cells for 5 hours after which wells were washed once with saline and fed regular medium. Forty-eight hours after transfection, cells were washed twice with phosphate-buffered saline, fixed in 1.25% glutaraldehyde for 5 minutes, and then incubated in a solution containing 2.5 mM of potassium ferrocyanide and potassium ferricyanide (Sigma Chemical Company, St. Louis, Mo.) and 0.5 mg/mL of 5-bromo-4-chloro-3-indolyl-β-D-galactoside (Life Technologies Corporate, Gaithersburg, Md.) at 37° C. for 4 hours. Cells with blue-stained nuclei will be scored as positive. Transfection efficiency was expressed as the percentage of positive staining cells to total cell count.

The in vitro ability of Ad-ER to infect and kill HLC cells were assessed. The cells were grown to 50% confluence in triplicates in 35-mm wells. The Ad-ER virus stock dilution that optimally infected HLC in the above experiment was used to infect the cells. The virus was left in contact with the cells for 5 hours after which wells were washed once with saline and fed regular medium. The cells were observed daily and the number of viable cells counted by trypan blue (Sigma, St. Louis, Mo.) exclusion test and hemocytometer.

### Example 6

### Study of Apoptosis Pathway in Adenovirus-Transfected Human Leiomyoma Cells

Previous work suggested that dominant negative estrogen receptor induced apoptosis in pituitary prolactinoma cell lines (Lee et al., 2001). Therefore the expression of two apoptosis-associated proteins BAX and Bcl-2 were tested by western blotting. Apoptosis is inhibited by the Bcl-2/Ced-9 family of proteins (Raff, 1992). The bcl-2 gene is overexpressed in many tumors including uterine fibroids (Matsuo et al., 1997).

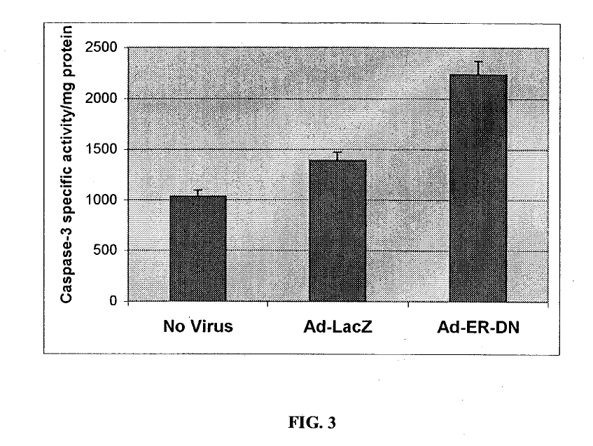

Human leiomyoma cells were plated in 10-cm culture dishes at a density of 5×10⁶ cells/dish. The following day, they were infected with adenoviral vectors at an MOI of 5 PFU/cell for 5 hours. After the addition of fresh medium, the cells were incubated for 48 or 72 hours. Cells were washed twice with PBS, and whole cell lysates prepared with lysis buffer (25% glycerol, 0.5 m NaCl, 1.5 mm MgCl₂, 20 mm HEPES [pH 7.9], 1 mm phenylmethylsulfonylfluoride, 0.2 mm EDTA, 25 mm NaF, and protease inhibitor cocktail tablets [Roche Molecular Biochemicals]). Equal amounts of protein (20 μg) was resolved by SDS-PAGE on 10% gel and transferred to nitrocellulose paper. The membranes were blocked with 3% nonfat milk in PBS for 1.5 hours and then incubated overnight at 4° C. with primary antibodies. Mouse monoclonal anti-Bcl-2 (1:1000; Santa Cruz Biotechnology, Inc) and mouse monoclonal anti-Bax (1:1000; Santa Cruz Biotechnology, Inc), were used for the detection of these two apoptosis-associated proteins. After three washes in 0.1% Tween-20 in PBS, immunoreactive proteins were detected using an antimouse or rabbit horseradish peroxidase-conjugated antibody (1:5000; Promega Corp) and the enhanced chemiluminescence system (Amersham Pharmacia Biotech, Arlington Heights, Ill.). Bands were detected with X-Omat film (Eastman Kodak Co., Rochester, N.Y.). Treatment with Ad-DNER increased Bax expression and decreased Bcl-2 expression. Caspase-3, an effector of apoptosis that causes degradation of structural and nuclear proteins, showed significantly higher levels one day after viral infection (FIG. 3).

### Example 7

### Study of the Bystander Phenomenon in Human Leiomyoma Cells Infected with Ad-ER

“Bystander effect” is a phenomenon in which cells infected with certain therapeutic gene will not only die but will also mediate killing of surrounding cells. This phenomenon was described in detail with the suicide gene therapy approach using thymidine kinase/ganciclovir (Freeman et al., 1993; Borrelli et al., 1988). This is an essential phenomenon in tumor gene therapy because currently, it is impossible to achieve 100% gene transfer in vivo. If the bystander phenomenon is operational in human leiomyoma cells infected with Ad-ER, this would suggest greater chances of success when this vector is used in clinical trials. It would mean that only a fraction of leiomyoma cells need be infected in an established fibroid tumor, but a major part of the tumor will undergo cell death. To study this phenomenon, cells were divided into two populations: one transfected with Ad-ER and the other transfected with Ad-LacZ. After transfection, the two populations of cells were cocultured at percentages of 0%, 25%, 50%, 75%, and 100% of Ad-ER transfected cells and plated in six-well plates and incubated for 5 days. The viability of the cells was determined using trypan blue exclusion method.

### Example 8

### In Vitro Transfection of Rat Leiomyoma Cell Line with Ad-ER Virus

A leiomyoma cell line, ELT 3 derived from the Eker rat was used. These cell lines are typical benign fibroid cell lines showing characteristic of smooth muscle tumors, and they also maintain expression of the estrogen receptor (Howe et al., 1995). ELT3 was maintained at 37° C. in 5% CO₂/air in medium 100/MCDB 105, supplemented with 5% fetal bovine serum.

The in vitro ability of Ad-ER to infect and kill ELT3 cells was assessed as described earlier (Al-Hendy et al., 2000; Al-Hendy and Auersperg, 1997). The cells were grown to 50% confluence in triplicates in 35-mm wells. The Ad-ER virus stock was diluted to 1, 10, 100, or 500 PFU/cell in culture medium and 1 mL of the virus suspension was placed in each well after removal of the medium. The virus was left in contact with the cells for 5 hours after which wells were washed once with saline and fed regular medium. The percentage of viable cells was measured using trypan blue exclusion test.

### Example 9

### In vivo Treatment of Uterine Fibroid—Mouse Model

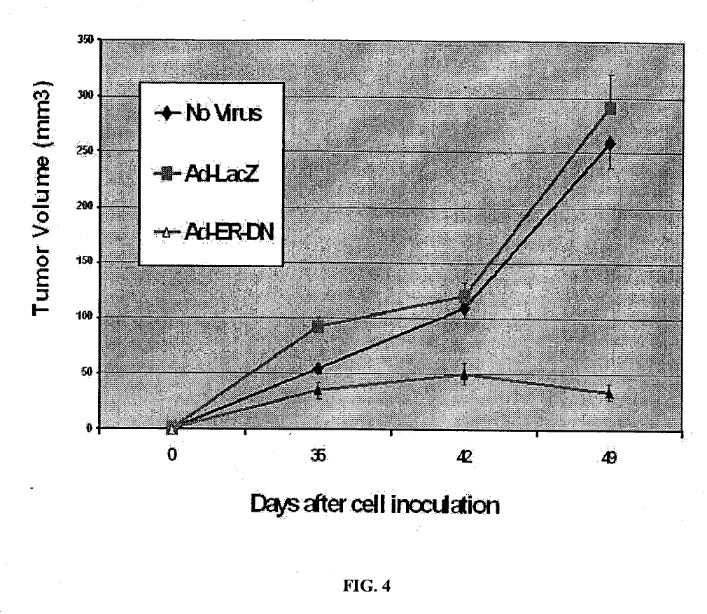

The nude mice-based animal model for uterine fibroid described previously was utilized (Howe et al, 1995) first to test the ability of Ad-ER to inhibit tumor formation in vivo (FIG. 4) and second to treat pre-established tumors.

Female BALB/c nude mice, 3 to 4 weeks old were purchased from Harlan Sprague Dawley (Indianapolis, Ind.) and hosted in the nude mice animal facilities with free access to food and water. Mice were subcutaneously implanted with 60-d estrogen pellets (Innovative Research of America, Sarasota, Fla.) 3 days before ELT3 cells implantation. Rat ELT3 cells were infected with the optimal MOI of adenoviruses to attain 100% transfection and incubated at 37° C. for 24 hours. Cells were collected, washed twice with PBS, resuspended in medium, and injected (2×10⁶ cells/mouse) into the flanks of the nude mice. The mice were divided into three groups each with 8 mice: group A, no virus; group B, Ad-LacZ; and group C, Ad-ER. Animals were examined for tumor formation every 2 days, and the size of the tumor was measured with calipers in three dimensions. Tumor size (cubic millimeters) was calculated using the formula:(3.14×length×width×depth)/6. The experiment was terminated when mice began to show morbidity or 6 weeks after cell implantation as per approved animal protocol.

### Example 10

### Direct Intratumor Injection of AdER-DN in BALB/c Nude Mouse Model

Material and Methods

Animal experiments. 6-8-week-old athymic female nude mice were ordered from Harlan—Sprague Dawley (Indianapolis, Ind). Three days after arrival, the mice were surgically implanted under neck skin with a 60-d estrogen pellet (17β-estradiol 0.075 mg) (Innovative Research of America Sarasota, Fla). One week later, all mice were injected in the right flank region with (5×10⁶) ELT3 cells (rat leiomyoma cells). Animals were examined weekly for tumor growth and the size of the tumors were measured with calipers in three dimensions. The volume of the tumors was calculated from the equation R1×R2×R3×0.5326. When tumor volume ranged between 50 to 100 mm,³ all mice were randomized into three treatment groups: Group 1 received no virus; Group 2, Ad-Lac-Z; and Group 3, AdER-DN. Groups 2 and 3 received 100 PFU/cell. All injections were performed directly into the four quadrants of tumors via separate entries. Tumors continued to be measured on a weekly basis. The experiment was terminated after approximately 2 months, when tumor volumes in control groups exceeded 30% of total body volume (in accordance with IACUC guidelines). Sample animals from all groups were sacrificed at different time points to study tumor response to different treatment options.

BrdU Assay. Two hours before sacrifice, mice from each group were injected intraperitoneally with 5-bromo-2′-deoxyuridine (BrdU) (no. B-5002, Sigma Chemical Co, St. Louis, Mo.) at 100 mg/kg from a 20 mg/mL stock. Animals were euthanized by CO₂. Representative sections of fibroid tumors were collected and fixed in 10% neutral-buffered formalin for 24 hours, then placed in 70% ETOH and processed for BrdU staining using anti-BrdU antibody (Becton Dickinson, Lincoln Park, NJ). The proliferative rates of tissues were assessed by counting the number of BrdU-positive tumor cells as a percent of the total number of cells in multiple random sections.

Tunel Assay. Formalin-fixed leiomyoma sections were processed using the DeadEnd Fluorometric Tunnel System Kit from Promega Corp (Madison, Wis.) following manufacturer instructions. Images were analyzed using a Nikon Microphot-fxa microscope, (Fuji, Tokyo 100, Japan) and Ektapress color film 800. Quantitation of cell death rates was determined as described for BrdU staining. Areas of obvious necrosis or infarction were excluded from analysis.

Statistical analysis. Statistical analysis was conducted using a statistical software package (SigmaStat; Jandel Scientific Inc, Chicago, Ill.). The differences in cell viability between different experimental groups and in the nude mice experiments were analyzed using two-way analysis of variance and pairwise comparison using the Student-Newman-Keuls method.

### Example 11

### In Vivo Treatment of Uterine Fibroid by Direct Intratumor Injection in BALB/c Nude Mouse Model

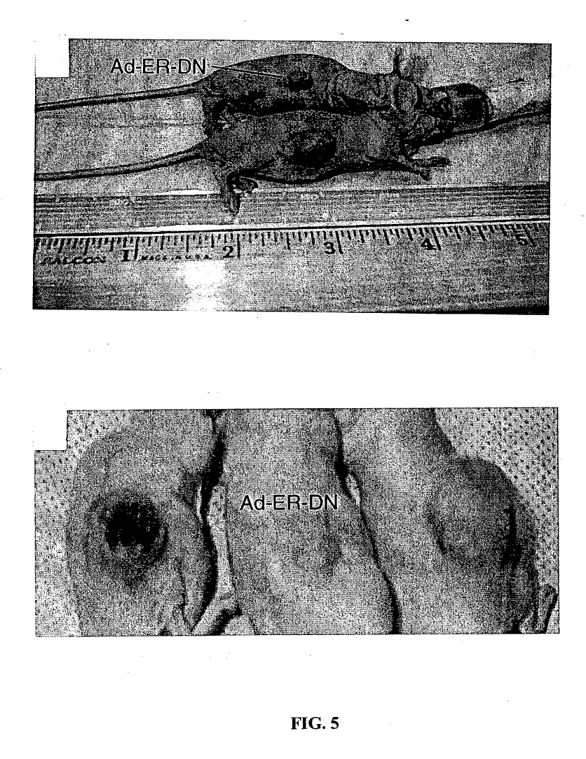

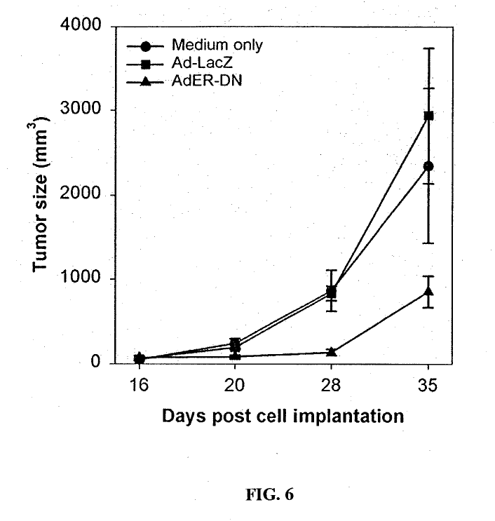

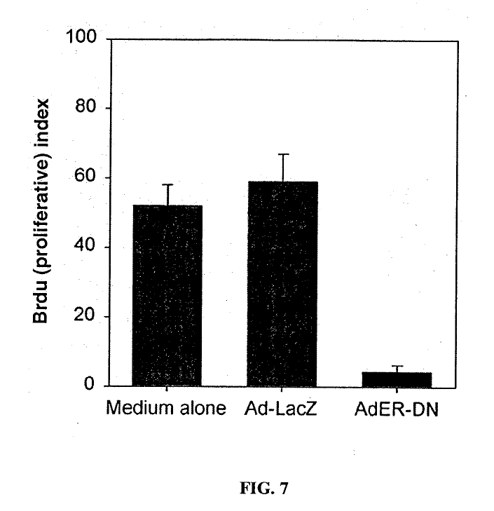

The methods and materials of the previous example showed that direct intratumoral injection of adenovirus was uneventful and well tolerated by all mice. The AdER-DN-treated mice demonstrated immediate overall arrest of tumor growth, with evident tumor regression in some mice (FIGS. 5 and 6). During the same time period, the tumors in the control groups continued to grow exponentially, increasing their volume by 500%-800% within 2 weeks. The difference in fibroid volume between AdER-DN-treated mice and control groups was highly significant (P=0.007). The AdER-DN-treated tumors demonstrated severely-inhibited cell proliferation (BrdU index=4.4%±2%) compared with control groups (52%±6% and 59%±8%, P<0.0001); see FIG. 7.

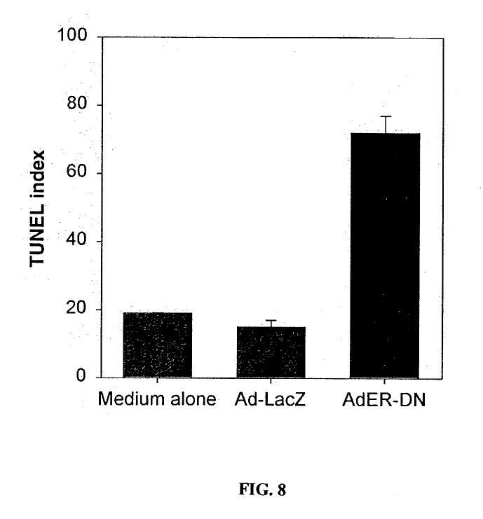

Additionally, there was a marked increase in the number of apoptotic cells in AdER-DN-treated fibroids (TUNEL index=71%±5%) versus control groups (19%±0.2%, and 15%±2%, P<0.0001); see FIG. 8.

Overall, the present invention demonstrates the ability of dominant-negative estrogen receptors to arrest and regress the growth of pre-established subcutaneous fibroids in a nude mouse model. This effect is mediated via induction of apoptosis and inhibition of cell proliferation.

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,554,101

U.S. Pat. No. 4,683,202

U.S. Pat. No. 5,466,468

U.S. Pat. No. 5,846,225

U.S. Pat. No. 5,846,233

U.S. Pat. No. 5,928,906

Al-Hendy and Auersperg, Gynecol Obst Invest., 43:268-275, 1997.

Al-Hendy et al., Am J Obstet Gynecol., 182:553-559, 2000.

Angel et al., Mol. Cell. Biol., 7:2256, 1987.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Baichwal and Sugden, In: Gene Transfer, Kucherlapati (ed.), New York, Plenum Press, 117-148, 1986.

Banerji et al., Cell, 27(2 Pt 1):299-308, 1981.

Banerji et al., Cell, 33(3):729-740, 1983.

Berkhout et al., Cell, 59:273-282, 1989.

Bett et al., J. Virol., 67:5911-5921, 1993.

Blanar et al., EMBO J., 8:1139, 1989.

Bodine and Ley, EMBO J., 6:2997, 1987.

Borrelli et al., Proc Natl Acad Sci USA, 85:7572-7576, 1988.

Boshart et al., Cell, 41:521, 1985.

Bosze et al., EMBO J, 5(7):1615-1623, 1986.

Braddock et al., Cell, 58:269, 1989.

Bradley et al., Br J Obstetr Gynecol., 105:235-240, 1998.

Bulla and Siddiqui, J. Virol., 62:1437, 1986.

Buttram and Reiter et al., Fertil Steril, 36:433 445, 1981.

Calson et al., N Engl J Med., 328:856-860, 1993.

Campbell and Villarreal, Mol. Cell. Biol., 8:1993, 1988.

Campere and Tilghman, Genes and Dev., 3:537, 1989.

Campo et al., Nature, 303:77, 1983.

Carter and Flotte et al., Curr Top Microbiol Immunol, 218:119-144, 1996.

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chattejee et al., Proc Natl. Acad. Sci. U.S.A., 86:9114, 1989.

Chen et al., Proc Natl Acad Sci USA, 91:3054-3057, 1994.

Chien et al., Mol Endocrinol., 13:2122-2136, 1999.

Choi et al., Cell, 53:519, 1988.

Chryssikopoulos and Loghis, Int Surg., 71:188, 1986.

Coffin, Retroviridae and Their Replication. In: Virology, Fields et al., eds., Raven Press, New York, pp. 1437-1500, 1990.

Cohen et al., J. Cell. Physiol., 5:75, 1987.

Costa et al., Mol. Cell. Biol., 8:81, 1988.

Coupar et al., Gene, 68:1-10, 1988.

Cripe et al., EMBO J., 6:3745, 1987.

Culotta and Hamer, Mol. Cell. Biol., 9:1376, 1989.

Culver et al., Science, 256(5063):1550-1552, 1992.

Dandolo et al., J. Virology, 47:55-64, 1983.

De Villiers et al., Nature, 312(5991):242-246, 1984.

Deschamps et al., Science, 230:1174-1177, 1985.

Edbrooke et al., Mol. Cell. Biol., 9:1908, 1989.

Edlund et al., Science, 230:912-916, 1985.

Englund et al., J Clin Endocrinol Metab., 83:92-96, 1998.

Feng and Holland, Nature, 334:6178, 1988.

Ferrari et al., J Virol, 70(5):3227-3234, 1996.

Firak and Subramanian, Mol Cell. Biol., 6:3667, 1986.

Fisher et al., Hum Gene Ther, 7(17):2079-87, 1996.

Flotte et al., Proc. Nat'l Acad. Sci. USA, 90(22):10613-7, 1993.

Foecking and Hofstetter, Gene, 45(1):101-105, 1986.

Freeman et al., Cancer Res., 53:5274-5283, 1993.

Fujimoto et al., Mol. Hum. Reprod., 5:742-747, 1999.

Fujita et al., Cell, 49:357, 1987.

Gilles et al., Cell, 33:717, 1983.

Gloss et al., EMBO J., 6:3735, 1987.

Godbout et al., Mol. Cell. Biol., 8:1169, 1988.

Goodbourn and Maniatis, Proc. Nat'l Acad. Sci. USA, 85:1447, 1988.

Goodbourn et al., Cell, 45:601, 1986.

Goodman et al., Blood, 84(5):1492-500, 1994.

Goodwin and Walker, Curr Opin Obstet Gynecol. 10:315-320, 1998.

Graham et al., In: Methods in Molecular Biology: Gene Transfer and Expression Protocols, ed. Murrary, E. J. (Humana, Clifton, N.J.), vol.7, 1991.

Greene et al., Immunology Today, 10:272, 1989

Grosschedl and Baltimore, Cell, 41:885, 1985.

Hallenbeck and Stevenson, Adv Exp Med Biol., 465:37-46, 2000.

Haslinger and Karin, Proc. Nat'l Acad. Sci. USA, 82:8572, 1985.

Hauber and Cullen, J. Virology, 62:673, 1988.

Hay et al., J. Mol Biol, 175(4):493-510, 1984.

Hearing and Shenk, J. Mol. Biol., 167(4):809-822, 1983.

Hearing et al., J. Virol., 61(8):2555-25558, 1987.

Hen et al., Nature, 321:249, 1986.

Hensel et al., Lymphokine Res., 8:347, 1989.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Howe et al., Am J Path., 146:1568-1579, 1995.

Howe et al., Endocrinology, 136:4996-5003, 1995.

Huang et al., Cell, 27:245, 1981.

Hug et al., Mol. Cell. Biol., 8:3065, 1988.

Hutchins and Berkowitz, J Am Assoc Gynecol Laparsc., 6:279, 1999.

Hwang et al., Mol. Cell. Biol., 10:585, 1990.

Ichimura et al., Fertil Steril., 70:967-971, 1998.

Imagawa et al., Cell, 51:251, 1987.

Imbra and Karin, Nature, 323:555, 1986.

Imler et al., Mol. Cell. Biol, 7:2558, 1987.

Imperiale and Nevins, Mol. Cell. Biol., 4:875, 1984.

Ince et al., Endocrinology, 136:3194-3199, 1995.

Ince et al., J Biol Chem., 268:14026-14032, 1993.

Inouye and Inouye, Nucleic Acids Res., 13: 3101-3109, 1985.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Johnson et al., In: Biotechnology And Pharmacy, Pezzuto et al., eds., Chapman and Hall, New York, 1993.

Johnson et al., Mol. Cell. Biol., 9:3393, 1989.

Kadesch and Berg, Mol. Cell. Biol., 6:2593, 1986.

Kaplitt, Nat Genet., 8(2):148-54, 1994.

Karin et al., Mol. Cell. Biol., 7:606, 1987.

Karin et al., Mol. Cell. Biol., 7:606, 1987.

Katinka et al., Cell, 20:393, 1980.

Kawamoto et al., Mol. Cell. Biol., 8:267, 1988.

Kessler et al., Proc Natl Acad Sci USA, 93(24):14082-14087, 1996.

Kiledjian et al., Mol. Cell. Biol., 8:145, 1988.

Klamut et al., Mol. Cell. Biol., 10:193, 1990.

Koch et al., Mol. Cell. Biol., 9:303, 1989.

Koeberl et al., Proc Natl Acad Sci USA, 94(4): 1426-31, 1997.

Kozarsky and Wilson, Curr Opin Genet Dev., 3:499-503, 1993.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984.

Kriegler et al., Cell, 53:45, 1988.

Kuhl et al., Cell, 50:1057, 1987.

Kunz et al., Nucl. Acids Res., 17:1121, 1989.

Kyte and Doolittle, J. Mol Biol, 57(1):105-32, 1982.

Larsen et al, Proc Natl. Acad. Sci. USA., 83:8283, 1986.

Laspia et al., Cell, 59:283, 1989.

Latimer et al., Mol. Cell. Biol., 10:760, 1990.

Lazennec et al., Mol Endocrinol., 13:969-980, 1999.

Lee et al., Endocrinology, 142:3756-3763, 2001.

Lee et al., Nature, 294:228, 1981.

Lee et al., Nucleic Acids Res., 12:4191-206, 1984.

Levinson et al., Nature, 295:79, 1982.

Levrero et al., Gene, 101: 195-202, 1991.

Lin et al., Mol. Cell. Biol., 10:850, 1990.

Lubahn et al., Proc. Natl. Acad. Sci. USA, 90:11162-11166, 1993.

Luria et al., EMBO J., 6:3307, 1987.

Lusky and Botchan, Proc Natl. Acad. Sci. U.S.A., 83:3609, 1986.

Lusky et al, Mol. Cell. Biol. 3:1108, 1983.

Majors and Varmus, Proc. Nat'l Acad. Sci. USA, 80:5866, 1983.

Mann et al., Cell, 33:153-159, 1983.

Matsuo et al., J Clin Endocrinol Metab. 82:293-299, 1997.

Matsuzaki et al., Mol. Hum. Reprod., 5:559-564, 1999.

McCown et al., Brain Res, 713(1-2):99-107, 1996.

McNeall et al., Gene, 76:81, 1989.

Miksicek et al., Cell, 46:203, 1986.

Mizukami et al., Virology, 217(1):124-130, 1996.

Mordacq and Linzer, Genes and Dev., 3:760, 1989.

Moreau et al., Nucl. Acids Res., 9:6047, 1981.

Muesing et al, Cell, 48:691, 1987.

Ng et al., Nuc. Acids Res., 17:601, 1989.

Nicolas and Rubinstein, In: Vectors: A survey of molecular cloning vectors and their uses, Rodriguez and Denhardt, eds., Stoneham: Butterworth, pp. 494-513, 1988.

Niu et al., Obstet Gynecol. 91:735-740, 1998.

Ondek etal., EMBO J., 6:1017, 1987.

Orimo et al, Proc. Natl. Acad. Sci. USA, 96:12027-12032, 1999.

Ornitz et al., Mol Cell. Biol., 7:3466, 1987.

Palmiter et al., Nature, 300:611, 1982.

Palomba et al., Fertil Steril., 76:38-43, 2001.

Paskind et al., Virology, 67:242-248, 1975.

Pech et al., Mol. Cell. Biol., 9:396, 1989.

Pelletier et al., Biol. Reprod., 61:1249-1255, 1999.

Perez-Stable and Constantini, Mol. Cell. Biol., 10:1116, 1990.

Picard and Schaffner, Nature, 307:83, 1984.

Ping et al., Microcirculation, 3(2):225-228, 1996.

Pinkert et al., Genes and Dev., 1:268, 1987.

Ponta et al., Proc. Nat'l Acad. Sci. USA, 82:1020, 1985.

Porton et al., Mol. Cell. Biol., 10: 1076, 1990.

Queen and Baltimore, Cell, 35:741, 1983.

Quinn et al., Mol. Cell. Biol., 9:4713, 1989.

Raff, Nature, 356:397-400, 1992.

Rauk et al., Am J Obstet Gynecol., 173:571-577, 1995.

Ravina et al., Contracept Fertil Sex, 23:45-49, 1995.

Redondo et al., Science, 247:1225, 1990.

Rein et al., Am J Obstet Gynecol., 172:14-18, 1995.

Reisman and Rotter, Mol. Cell. Biol., 9:3571, 1989.

Remington's Pharmaceutical Sciences, 15^{th} ed., pages 1035-1038 and 1570-1580, Mack Publishing Company, Easton, Pa., 1980.

Resendez Jr. et al., Mol. Cell. Biol., 8:4579, 1988.

Ridgeway, In: Vectors: A survey of molecular cloning vectors and their uses. Rodriguez and Denhardt, eds. Stoneham: Butterworth, pp. 467-492, 1988.

Ripe et al., Mol. Cell. Biol., 9:2224, 1989.

Rittling et al., Nuc. Acids Res., 17:1619, 1989.

Rosen et al., Cell, 41:813, 1988.

Roux et al, Proc. Nat'l Acad. Sci. USA, 86:9079-9083, 1989.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Samulski et al., J. Virol., 61(10):3096-3101, 1987.

Satake et al., J. Virology, 62:970, 1988.

Sauders et al., J. Endocrinol., 154:R13-R16, 1997.

Schaffner et al., J. Mol. Biol., 201:81, 1988.

Searle et al., Mol. Cell. Biol., 5:1480, 1985.

Sener et al., Fertil Steril., 65:354-357, 1996.

Sharp and Marciniak, Cell, 59:229, 1989.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takebe et al., Mol. Cell. Biol., 8:466, 1988.

Tavernier et al., Nature, 301:634, 1983.

Taylor and Kingston, Mol. Cell. Biol., 10: 165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Thiesen et al., J. Virology, 62:614, 1988.

Tibbetts Cell, 12:243-249, 1977.

Treisman, Cell, 42:889, 1985.

Tronche et al., Mol. Biol. Med., 7:173, 1990.

Trudel and Constantini, Genes and Dev. 6:954, 1987.

Tyndell et al., Nuc. Acids. Res., 9:6231, 1981.

Vannice and Levinson, J. Virology, 62:1305, 1988.

Vasseur et al., Proc Natl. Acad. Sci. U.S.A., 77:1068, 1980.

VeKaut, Curr Opin Obstet Gynecol., 5:301, 1993.

Vilos et al., J. Am Assoc Gynecol Laparsc., 5:289-292, 1998.

Vilos, Can J Diagnosis., 17:55-59, 2000.

Vilos, Canadian J. Diagnosis, 14:145-151, 1997.

Vollenhoven et al., Br J Obstet Gynecol., 97:255-298, 1990.

Wada et al, Nucleic Acids Res. 18:2367-2411, 1990.

Wang and Calame, Cell, 47:241, 1986.

Wang et al., Biol. Reprod., 61:955-964, 1999.

Weber et al., Cell, 36:983, 1984.

Weinberger et al. Mol. Cell. Biol., 8:988, 1984.

Winoto and Baltimore, Cell 59:649, 1989.

Wu et al., Eur. J. Endocrinol., 142:92-99, 2000.

Xiao, et al., J. Virol., 70:8098-8108, 1996.

Yutzey et al. Mol. Cell. Biol., 9:1397, 1989.

What is claimed is:

1. A method for treating an estrogen-dependent genitourinary condition in a patient comprising administering to the patient an effective amount of an expression construct comprising a nucleic acid comprising a sequence encoding a modified estrogen receptor, wherein the sequence is under the control of a promoter.

2. The method of claim 1, wherein the genitourinary condition is a condition of the uterus.

3. The method of claim 2, wherein the condition is a leiomyoma, adenomyosis, endometriosis, endometrial hyperplasia, leiomyosarcoma, dysfunctional uterine bleeding, or cancer.

4. The method of claim 3, wherein the condition is a leiomyoma.

5. The method of claim 4, wherein the leiomyoma is a submucous, intramural, or subserous fibroid.

6. The method of claim 1, further comprising identifying a patient in need of the treatment.

7. The method of claim 6, wherein the patient is identified by detecting a leiomyoma in the patient.

8. The method of claim 1, wherein the modified estrogen receptor is a modified estrogen receptor α

9. The method of claim 1, wherein the modified estrogen receptor is a modified estrogen receptor β.

10. The method of claim 1, wherein the expression construct is a viral vector.

11. The method of claim 10, wherein the viral vector is an adenovirus vector, an adeno-associated virus vector, a herpesvirus vector, a lentivirus vector, a retrovirus vector, or a vaccinia virus vector.

12. The method of claim 11, wherein the viral vector is an adenovirus vector.

13. The method of claim 12, wherein the expression construct is Ad-ER1-536.

14. The method of claim 11, wherein the patient is administered about 10³ to about 10¹⁵ viral particles.

15. The method of claim 1, wherein the construct is administered to the patient intrauterinely, intravaginally, intravenously, directly to the affected area, intraperitoneally, or regionally.

16. The method of claim 15, wherein the construct is administered intrauterinely to the patient via a catheter.

17. The method of claim 15, wherein the construct is administered directly to the affected area by injection.

18. The method of claim 1, wherein the construct is administered to the patient more than one time.

19. The method of claim 1, wherein the modified estrogen receptor has a mutation that affects DNA binding activity, transcriptional activation activity, dimerization activity, ligand binding activity, growth hormone binding activity, or binding activity to AP-1 or to a component of AP-1.

20. The method of claim 19, wherein the mutation is a point mutation or deletion.

21. The method of claim 20, wherein the mutation is a point mutation.

22. The method of claim 21, wherein the point mutation is a deletion, a substitution, or an insertion mutation.

23. The method of claim 22, wherein the mutation is at amino acid 540, substituting a charged residue for an uncharged residue.

24. The method of claim 22, wherein the mutation inserts a frameshift at codon 554.

25. The method of claim 20, wherein the mutation is a deletion comprising at least 2 residues.

26. The method of claim 25, wherein the modified estrogen receptor is a truncated receptor.

27. The method of claim 26, wherein the truncated receptor lacks at least 20 contiguous amino acids of SEQ ID NO:2 or SEQ ID NO:4.

28. The method of claim 27, wherein the truncated receptor is ER1-530 or ER1-536.

29. The method of claim 4, further comprising removing the leiomyona.

30. The method of claim 1, further comprising administering a second dominant negative estrogen receptor to the patient.

31. The method of claim 30, wherein modified estrogen receptors α and β are administered to the patient.

32. A method for inhibiting a leiomyoma cell comprising providing to the cell an effective amount of a dominant negative estrogen receptor, wherein the leiomyoma cell is inhibited.

33. The method of claim 32, wherein the dominant negative estrogen receptor is a dominant negative estrogen receptor α.

34. The method of claim 32, wherein the leiomyoma is a uterine leiomyoma.

35. The method of claim 32, wherein the modified estrogen receptor is provided to the cell by administering to the cell an adenovirus vector comprising a nucleic acid sequence, under the control of a promoter, encoding the modified estrogen receptor, wherein the modified estrogen receptor is expressed in the cell.

36. The method of claim 35, wherein the promoter is a CMV IE promoter.

37. The method of claim 36, wherein the adenovirus vector comprising the nucleic acid sequence, under the control of a promoter, encoding the modified estrogen receptor is Ad-ER1-536.

38. The method of claim 32, wherein the leiomyoma cell undergoes apoptosis.

39. The method of claim 32, wherein the leiomyoma cell is in a patient.

40. The method of claim 32, wherein the modified estrogen receptor has a mutation that affects DNA binding activity, transcriptional activation activity, dimerization activity, ligand binding activity, or growth hormone binding activity, binding activity to AP-1 or to a component of AP-1.

41. The method of claim 40, wherein the mutation is a point mutation or deletion.

42. The method of claim 41, wherein the mutation is a point mutation.

43. The method of claim 42, wherein the point mutation is a deletion, a substitution, or an insertion mutation.

44. The method of claim 43, wherein the mutation is a substitution.

45. The method of claim 44, wherein the mutation is at amino acid 540, substituting a charged residue for an uncharged residue.

46. The method of claim 43, wherein the point mutation inserts a frameshift.

47. The method of claim 41, wherein the mutation is a deletion comprising at least 2 residues.

48. The method of claim 61, wherein the modified estrogen receptor is a truncated receptor.

49. The method of claim 48, wherein the truncated receptor is ER1-530 or ER1-536.

50. The method of claim 48, wherein the truncated receptor lacks at least 20 contiguous amino acids of SEQ ID NO:2.

51. A method of treating a uterine fibroid in a patient comprising administering to the patient an effective amount of an adenovirus construct comprising a nucleic acid sequence, under the control of a promoter, encoding an estrogen receptor that is capable of binding to a ligand and has a reduced ability to activate transcription of an estrogen-dependent gene, wherein the fibroid is reduced.

52. The method of claim 51, wherein the construct is administered more than once.

53. The method of claim 51, wherein the estrogen receptor has a mutation in a transactivation domain, in a DNA binding domain, or in a region mediating protein-protein interaction.

54. A method of preventing pregnancy in a female subject comprising administering an effective amount of an expression construct comprising a nucleic acid, under the control of a promoter, encoding a modified estrogen receptor, wherein pregnancy is prevented.

55. The method of claim 54, wherein the expression construct is a viral vector.

56. The method of claim 54, wherein the modified estrogen receptor has a mutation that affects DNA binding activity, transcriptional activation activity, dimerization activity, ligand binding activity, or growth hormone binding activity, binding activity to AP-1 or to a component of AP-1.

57. The method of claim 54 wherein the modified estrogen receptor is a dominant-negative estrogen receptor.

58. The method of claim 57, wherein the dominant-negative estrogen receptor is ER1-536.

59. The method of claim 54, further comprising administering to a female subject a second agent for preventing conception.
